SEQUENCE LISTING (15 sequences)

SEQ ID NO: 1 - DNA, 25 bp, Artificial
  oligonucleotide
caccgatgggtgatgggagtccctg

SEQ ID NO: 2 - DNA, 25 bp, Artificial
  oligonucleotide
aaaccagggactcccatcacccatc

SEQ ID NO: 3 - DNA, 24 bp, Artificial
  oligonucleotide
caccgaagctcagccgcgcggcga

SEQ ID NO: 4 - DNA, 24 bp, Artificial
  oligonucleotide
aaactcgccgcgcggctgagcttc

SEQ ID NO: 5 - DNA, 25 bp, Artificial
  oligonucleotide
caccgcaaaggccaggatcaccagg

SEQ ID NO: 6 - DNA, 25 bp, Artificial
  oligonucleotide
aaaccctggtgatcctggcctttgc

SEQ ID NO: 7 - DNA, 24 bp, Artificial
  oligonucleotide
caccgatttcaaaagtgatcacca

SEQ ID NO: 8 - DNA, 24 bp, Artificial
  oligonucleotide
aaactggtgatcacttttgaaatc

SEQ ID NO: 9 - DNA, 1431 bp, Homo sapiens
atgggtcctggtccacctgccgctggcgcggctccatctccacggcccctgagtctcgtagcccggttgtcctatgccgtgggccatttccttaatgacctttgtgccagtatgtggtttacctacttgttgctttatctccatagcgtgagagcttattcttctcgaggagcgggtttgctcctgttgctgggccaagttgcagatggcctgtgcacccctcttgtgggttatgaggccgatagagcggcaagttgctgtgcaaggtatggtccgcgaaaggcatggcatttggttggcaccgtatgcgtacttctttctttcccatttatcttctctccatgtctcggctgcggcgcagccacccccgaatgggctgctttgttgtattacggacctttcatcgtaatattccaattcggctgggcgtcaacgcaaattagccatctcagtctcattccggaacttgttactaatgatcacgaaaaagtagagcttcgctatgccttcaccgttgtagccaacataacggtacacggcgctgcctggcttctcttgcacctccaggggagctcaagggtcgagccaacgcaggatatttcaatatcagaccagctcggtggtcaggatgttccggtgttccggaatctgagtttgctcgtcgtcggagttggagctgtcttcagccttcttttccaccttggaacaagggaacgcagacggcctcacgctgaagagccaggtgagcacactccgcttctggcccctgctacagcgcaacccttgcttctctggaagcattggctgagagaacccgctttctatcaagtgggcatactgtatatgacaacgaggcttattgtgaatctgagtcaaacctatatggccatgtatttgacatattctcttcacttgcctaaaaagttcatcgccacaattccgcttgttatgtatctgagtggtttcttgagtagctttctgatgaagccgatcaacaagtgcattggacggaacatgacgtacttcagtggccttttggtcatcctggcttttgcagcatgggttgctcttgcggagggcctgggagtagcagtgtatgcggctgctgttctgttgggggccgggtgtgcaacaatcctcgtgacgtcccttgcgatgacggcagatctgattgggcctcacacgaactccggagccttcgtttacggttctatgtccttcttggacaaggttgctaatgggcttgccgtgatggcaattcaatcccttcacccgtgcccttctgagttgtgctgcagagcgtgtgtgtccttttatcattgggctatggtcgctgtgacgggtggagtaggggtggcagcagccctctgcctctgtagtcttttgctgtggccgactaggcttcgccgctgggaccgggacgcccgcccg

SEQ ID NO: 10 - DNA, 21 bp, Artificial
  shRNA
cccattctcaactctaatcca

SEQ ID NO: 11 - DNA, 21 bp, Artificial
  shRNA
cttcttgtcctccttcctcat

SEQ ID NO: 12 - DNA, 20 bp, Artificial
  primer
tccacacagatctcccacct

SEQ ID NO: 13 - DNA, 19 bp, Artificial
  primer
gccgtagacggtgatgttg

SEQ ID NO: 14 - DNA, 18 bp, Artificial
  primer
ggttccgctgccctgagg

SEQ ID NO: 15 - DNA, 21 bp, Artificial
  primer
gaaggtagtttcgtggatgcc

(19) United States
(12) Patent Application Publication
(10) Pub. No.: US 2023/0103549 A1
(43) Pub. Date: Apr. 6, 2023

(54) OVERLAPPING THERAPEUTIC STRATEGIES FOR CYSTINOSIS TREATMENT AND COSMETIC SKIN DARKENING

(71) Applicant: Whitehead Institute for Biomedical Research, Cambridge, MA (US)

(72) Inventors: David M. Sabatini, Cambridge, MA (US); Charles H. Adelmann, Cambridge, MA (US); Anna K. Traunbauer, Cambridge, MA (US)

(21) Appl. No.: 17/771,808

(22) PCT Filed: Oct. 25, 2020
(86) PCT No.: PCT/US2020/057259

§ 371 Date: Apr. 25, 2022

Related U.S. Application Data
(60) Provisional application No. 63/001,140, filed on Mar. 27, 2020.
(60) Provisional application No. 62/926,320, filed on Oct. 25, 2019.

(51) Int. Cl. C12N 15/113; C12N 15/11; C12N 9/22; C12N 15/90; A61P 3/00

(52) U.S. Cl. CPC C12N 15/113; C12N 15/11; C12N 9/22; C12N 15/907; A61P 3/00; C12N 2310/20; C12N 2800/80; C12N 2310/14; C12N 2310/122; C12N 2310/531

(57) ABSTRACT

Disclosed herein are methods and compositions for modulating MFSD12 expression and activity to treat diseases such as lysosomal storage diseases, including cystinosis. Also disclosed are methods of altering skin pigmentation and methods of screening for MFSD12 modulation agents.

### CROSS-REFERENCE TO RELATED APPLICATIONS

This application claims the benefit of U.S. Provisional Application No. 62/926,320, filed on Oct. 25, 2019 and U.S. Provisional Application No. 63/001,140, filed on Mar. 27, 2020. The entire teachings of the above applications are incorporated herein by reference.

### BACKGROUND OF THE INVENTION

Occasionally a therapeutic strategy employed for treating a condition and/or disease may also be identified as providing non-medical benefits and/or uses. For example, a therapeutic strategy may be developed for treating both a rare disease, as well as providing possible cosmetic benefits.

Cystinosis is a rare genetic disease occurring in approximately 1/100K live births. The current standard of care for cystinosis, oral cysteamine, has a short half-life and uncomfortable side-effects which are documented barriers for patient compliance. In addition, sunless tanning is currently a $1 billion industry that continuously grows as the dangers of UV exposure used in conventional tanning are better realized by the general population. Current commercially available sunless tanning options are chemical dyes that often produce skin colorations that are unnatural in appearance. In addition, new therapies for activating the endogenous melanin synthesis pathway coincidently activate melanocyte differentiation pathways that are implicated in driving melanomas.

Thus, there is a need for a cystinosis therapy that has fewer side effects and a longer half-life, and there also continues to be a demand for improved sunless tanning options that are not harmful and provide the desired cosmetic benefits. A therapeutic strategy has been developed that is applicable to both treating rare diseases, as well as providing non-medical uses (e.g., cosmetic uses).

### SUMMARY OF THE INVENTION

MFSD12 is a promising target for cystinosis therapy and cosmetic skin darkening. Disclosed herein are data showing inhibition of the MFSD12 protein's cysteine transport function in the lysosome could be used to treat cystinosis, a debilitating lysosomal storage disorder. A new therapeutic target like MFSD12 would provide a better therapy for cystinosis patients. As the MFSD12 KO mouse appears phenotypically normal, it is expected that targeting MFSD12 results in few major side-effects.

Additionally, the present invention shows that cysteine import by MFSD12 is the mechanism through which MFSD12 is required for pheomelanin synthesis in melanocytes. Topical application of an MFSD12 inhibitor would darken skin by inhibiting pheomelanin synthesis. The therapy detailed here would be downstream of melanocyte differentiation and function through boosting the production of already occurring pigment synthesis pathways.

The present invention also provides methods for assaying MFSD12 function in vitro, which could be used to develop MFSD12 inhibitors for both cystinosis treatment and for cosmetic darkening of the skin.

Some aspects of the disclosure are related to a method of modulating or stabilizing cysteine transport function in a lysosome and/or in a melanosome of a cell, comprising modulating the expression of MFSD12 or the activity of a gene product of MFSD12 in the cell. In some embodiments, the expression of MFSD12 or the activity of a gene product of MFSD12 is increased, thereby increasing the level of cysteine in the lysosome and/or in a melanosome. In some embodiments, the expression of MFSD12 or the activity of a gene product of MFSD12 is decreased, thereby decreasing the level of cysteine in the lysosome and/or in a melanosome. In some embodiments, the level of cysteine in the lysosome and/or in a melanosome is stabilized. In some embodiments, the level of cysteine in the lysosome and/or in a melanosome is decreased (e.g., by at least 95%, by at least 85%, or by at least 75%). In some embodiments, the expression of MFSD12 or the activity of a gene product of MFSD12 is modulated by contacting the cell with an agent. In some embodiments, the agent comprises a polypeptide, amino acid, oligonucleotide, lipid, carbohydrate, hybrid molecule, peptide, nucleic acid (e.g., siRNA, shRNA, antisense oligonucleotide, aptamer, or random oligonucleotide), or small molecule. In some embodiments, the expression of MFSD12 or the activity of a gene product of MFSD12 is modulated using gene editing (e.g., CRISPR, TALEN, or ZFN).

Some aspects of the disclosure are related to a method of treating or preventing a disease or disorder associated with an aberrant level of cysteine/cystine, in a lysosome and/or in a melanosome of a subject, comprising administering to the subject an agent that modulates the expression of MFSD12 or the activity of a gene product of MFSD12. In some aspects, cysteine is present in the form of cystine.

In some embodiments, administration of the agent increases the level of cysteine in lysosomes and/or melanosomes of the subject. In some embodiments, administration of the agent decreases the level of cysteine in lysosomes and/or melanosomes of the subject. In some embodiments, administration of the agent stabilizes the level of cysteine in lysosomes and/or melanosomes of the subject. In some embodiments, the agent comprises a polypeptide, amino acid, oligonucleotide, lipid, carbohydrate, hybrid molecule, peptide, nucleic acid (e.g., siRNA, shRNA, antisense oligonucleotide, aptamer, or random oligonucleotide), or small molecule. In some embodiments, the expression of MFSD12 or the activity of a gene product of MFSD12 is modulated using gene editing (e.g., CRISPR, TALEN, or ZFN). In some embodiments, the disease or disorder is a lysosomal storage disease or disorder. In some embodiments, the disease or disorder is cystinosis. In some embodiments, the method further comprises administration of a second agent (e.g., cysteamine) to the subject.

Some aspects of the disclosure are related to a method of altering skin pigmentation of a subject, comprising topically administering to the subject an agent that modulates the expression of MFSD12 or the activity of a gene product of MFSD12 in the subject.

In some embodiments, administration of the agent inhibits pheomelanin synthesis. In some embodiments, administration of the agent decreases the level of cysteine in melanosomes of the subject. In some embodiments, the agent comprises a polypeptide, amino acid, oligonucleotide, lipid, carbohydrate, hybrid molecule, peptide, nucleic acid (e.g., siRNA, shRNA, antisense oligonucleotide, aptamer, or random oligonucleotide), or small molecule. In some embodiments, expression of MFSD12 or the activity of a gene product of MFSD12 is modulated using gene editing (e.g., CRISPR, TALEN, or ZFN). In some embodiments, the expression of MFSD12 or the activity of a gene product of MFSD12 is decreased, thereby darkening skin pigmentation of the subject.

Some aspects of the disclosure are related to a composition, comprising an agent that modulates the expression of MFSD12 or the activity of a gene product of MFSD12 when administered to a subject.

In certain aspects, the agent increases the expression of MFSD12 or the activity of a gene product of MFSD12. In some embodiments, the agent decreases the expression of MFSD12 or the activity of a gene product of MFSD12. In some aspects, the agent stabilizes the level of cysteine. In some embodiments, the agent comprises a polypeptide, amino acid, oligonucleotide, lipid, carbohydrate, hybrid molecule, peptide, nucleic acid (e.g., siRNA, shRNA, antisense oligonucleotide, aptamer, or random oligonucleotide), or small molecule. In some embodiments, the expression of MFSD12 or the activity of a gene product of MFSD12 is modulated using gene editing (e.g., CRISPR, TALEN, or ZFN). In some embodiments, the composition further includes a second agent (e.g., cysteamine). In some embodiments, the composition is a cosmetic composition. In some embodiments, the cosmetic composition is a topical cosmetic composition.

Some aspects of the disclosure are related to a method of identifying a candidate agent that modulates the expression of MFSD12 or the activity of a gene product of MFSD12 in a lysosome and/or in a melanosome, comprising contacting the lysosome and/or the melanosome with a test agent, measuring a level of cysteine in the lysosome and/or in the melanosome, and identifying the agent as an inhibitor of expression of MFSD12 or the activity of a gene product of MFSD12 if the level of cysteine in the lysosome and/or in the melanosome is lower than a reference level, or identifying the test agent as an agent that increases expression of MFSD12 or the activity of a gene product of MFSD12 if the level of cysteine in the lysosome and/or in the melanosome is higher than a reference level, wherein the reference level is the level of cysteine in lysosomes and/or in melanosomes under equivalent conditions but not exposed to the test agent.

In some embodiments, the agent comprises a polypeptide, amino acid, oligonucleotide, lipid, carbohydrate, hybrid molecule, peptide, nucleic acid, or small molecule. In some embodiments a nucleic acid used as a test agent comprises a siRNA, shRNA, antisense oligonucleotide, aptamer, or random oligonucleotide. In some embodiments, the expression of MFSD12 or the activity of a gene product of MFSD12 is modulated using gene editing (e.g., CRISPR, TALEN, or ZFN). In some embodiments, the test agent is capable of increasing the level of expression of MFSD12 or the activity of a gene product of MFSD12 by at least 10%, 25%, 75%, or 100%.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, ncbi.nlm.nih.gov/omim/ and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The above discussed, and many other features and attendant advantages of the present inventions will become better understood by reference to the following detailed description of the invention in conjunction with the attached drawings.

### BRIEF DESCRIPTION OF THE DRAWINGS

The patent or application file contains at least one drawing executed in color. Copies of this patent or patent application publication with color drawings will be provided by the Office upon request and payment of the necessary fee. FIGS. 11-20 re-present data from FIGS. 1-10, as well as providing additional data.

FIGS. 1A-1B demonstrates MFSD12 is required for pheomelanin synthesis. MFSD12 KO leads to reduced pheomelanin (red pigment) synthesis and darkened skin/fur. FIG. 1A provides a photograph of clonal wild-type and MFSD12 knock-out SKMEL30 cells showing decreased brown/red coloration. FIG. 1B shows Mfsd12 knockout agouti have no detectable red “pheomelanin” in their fur, while they still have brown/black “eumelanin” (Crawford et al., 2017).

FIG. 2 depicts a method for rapidly isolating melanosomes.

FIG. 3 shows MFSD12 is necessary for maintaining cystine levels in melanosomes. MelanolP was used to isolate melanosomes by tagging GPR143, a marker for melanosomes. Untargeted polar metabolite profiling of MelanolP isolates from wild-type and Mfsd12 knock-out B16-F10 cells, highlights the dependence of cystine levels on Mfsd12 (n=4).

FIG. 4A-4B shows cysteinylDOPA detection via metabolite profiling. Minor and major isoforms correspond to 2 and 5 cysteinylDOPA isoforms. FIGS. 4A-4B includes cysteinylDOPA levels in B16F10 melanoma and SKMEL30 melanoma cells. The method for detecting cysteinylDOPAs was adapted from Martin et al. “Development of a mass spectrometry method for the determination of a melanoma biomarker, 5-S-cysteinyldopa, in human plasma using solid phase extraction for sample clean-up.” J Chromatogr A. 2007 Jul. 13; 1156(1-2):141-8. Epub 2006 Dec. 29.

FIG. 5 illustrates a model for the effect of MFSD12 KO on pheomelanin synthesis.

FIG. 6 shows many melanosomal genes have expression restricted to pigmented tissues while MFSD12 is expressed ubiquitously (i.e., is expressed in both pigmented and non-pigmented tissues). FIG. 6 shows expression profiles of known pigment genes (TYR and SLC45A2) and MFSD12 from the FANTOM5 data set, accessed via Human Protein Atlas. Retina tissue (red) is highly enriched with retinal pigment epithelial cells, which synthesize melanosomes like melanocytes.

FIGS. 7A-7B demonstrate MFSD12 maintains cystine and cysteine in lysosomes. FIG. 7A shows untargeted polar metabolite profiling of LysolP (a rapid method for the immunopurification of lysosomes) isolates from wild-type and MFSD12 knock-out HEK293T cells, highlight the dependence of cystine levels on MFSD12 (n=6).

FIG. 7B shows targeted polar metabolite profiling of cystine and cysteine in whole cell and LysolP isolates from wild-type and MFSD12 knock-out cell lines. Cysteine was detected via derivatization with Ellman's reagent (mean±SEM, n=3, *P<0.05).

FIG. 8 shows MFSD12 is epistatic to CTNS, the lysosomal cystine exporter mutated in cystinosis. FIG. 8 shows targeted polar metabolite profiling of cystine in whole cell and LysolP isolates from wild-type, MFSD12 knock-out, CTNS knock-out, and MFSD12/CTNS double knock-out HEK293T cell lines (mean±SEM, n=3, *P<0.05) with linear axes.

FIGS. 9A-9C show MFSD12 is necessary for cysteine uptake in lysosomes in vitro and transports cysteine in an in vitro reconstitution system. FIG. 9A shows polar metabolite profiling of lysosomal extracts from in vitro uptake assays. After isolating lysosomes from wild-type or MFSD12 knock-out HEK293T cells, purified lysosomes were incubated in transport assay buffer supplemented with 100 μM [¹³C₃, ¹⁵Ni]-cysteine for the indicated time points. Control IPs were from cell lines expressing the FLAG-LysolP tag not expected to be precipitated by anti-HA beads. FIG. 9B shows a single time point uptake of 8 μM ¹⁴C₂ labeled cysteine by purified MFSD12 reconstituted into proteoliposomes. FIG. 9C shows polar metabolite profiling of lysosomal extracts from in vitro uptake assays. After isolating lysosomes from wild-type or MFSD12 knock-out HEK293T cells, lysosomes were isolated via differential centrifugation and normalized with a hexoaminidase assay. The uptake assay was performed in 1% BSA, KPBS, 125 mM sucrose, and 3 mM DTT. 35 μM cysteine substrate unless where specified.

FIGS. 10A-10B show [¹⁴C] melanosomal and lysosomal cellular uptake assays. FIGS. 10A-10B show 50e6 HEK 293T or B16F10 cells were incubated in 1.2 μM [¹⁴C]-cystine in HBSS before organelles were isolated via the LysolP or MelanolP, respectively. Lysosomal capture for LysolPs were normalized by hexoaminidase assay. MelanolP and LysolP methods demonstrate MFSD12 is required for acute uptake of ¹⁴C into these organelles as cystine is transported into the cell and presumably reduced to cysteine in the cytosol.

FIG. 11 shows MFSD12 regulates pigmentation and cysteine storage. FIG. 11 re-presents data from FIG. 5, as well as providing additional data.

FIGS. 12A-12B provide further data on the MelanolP. FIG. 12A includes immunostaining showing the MelanoTag colocalizing with Tyrp1, an established melanosomal marker (see also FIG. 14C). FIG. 12B shows immunoblots from B16F10 and SKMEL30 cells with the MelanolP. Additional method details regarding the LysolP can be found in Abu-Remaileh et al., “Lysosomal metabolomics reveals V-ATPase- and mTOR-dependent regulation of amino acid efflux from lysosomes.” Science. 2017 Nov. 10; 358(6364):807-813. Epub 2017 Oct. 26. FIGS. 12A-12B re-present data from FIG. 2, as well as providing additional data.

FIG. 13 shows MFSD12 knock-down rescues cystinosis phenotype in primary fibroblasts. Whole-cell cystine levels in fibroblasts from unaffected parent (GM00906) and cystinotic proband (GM00379) with lentiviral shRNA knock-down of MFSD12. Cell lines (n=4) are described in Zykovich et al., “CTNS mutations in publicly-available human cystinosis cell lines.” Molecular Genetics & Metabolism Reports, 5 (2015) 63-66.

FIGS. 14A-14E show MelanolP enables the rapid isolation of pure melanosomes. FIG. 14A provides schematic diagrams of the MelanoTag (GPR143-mScarlet-3xHA) and the MelanolP workflow. FIG. 14B shows immunofluorescence analyses of B16F10 cells expressing the MelanoTag using antibodies to Tyrp1 and the HA epitope tag. Cells were treated for two days with 10 μM forskolin before immunofluorescent labeling. TYRP1 and MelanoTag positive puncta are concentrated on the cell periphery, consistent with staining patterns observed previously for both markers (Bruder et al., 2012). FIG. 14C shows immunoblot analyses of whole-cell lysates, control immunoprecipitates (from cells expressing a myc-MelanoTag), and purified melanosomes (from cells expressing the HA-MelanoTag) prepared from murine B16F10 and human SKMEL30 cells. FIG. 14D shows untargeted polar metabolite profiling of melanosomes isolated from wild-type and Tyr knock-out cells. The blue datapoints highlight a candidate eumelanin synthesis intermediate, identified via ddMS2 spectra on Compound Discoverer, and the red ones all the detected proteogenic amino acids, including tyrosine, which is labelled (n=3 per condition). FIG. 14E shows metabolite profiling analysis of ‘Melanin Intermediate’ and tyrosine in whole-cell lysates and purified melanosomes from wild-type and Tyr knock-out B16F10 cells. This melanin intermediate has been annotated as indole-5,6-quinone, based on a mixed indole-5,6-quinone/dihydroxyindolquinone species observed in chemical standards (mean±SEM; n=3 per condition; * P<0.05, *** P<0.001).

FIGS. 15A-15G demonstrate MFSD12 is necessary to maintain melanosomal cystine levels and produce cysteinyldopas. FIG. 15A shows untargeted polar metabolite profiling of melanosomes isolated from Mfsd12 knock-out and wild-type B16F10 cells. The red datapoint highlights cystine (n=3 per condition). FIG. 15B shows follow-up metabolite analysis of cystine in B16F10 whole-cell lysates and purified melanosomes (mean±SEM; n=3; *** P<0.001). FIG. 15C shows metabolite profiling of cysteinyldopas in B16F10 whole-cell lysates. The ‘Minor Isomer’ and ‘Major Isomer’ are distinguished by retention time, and they are annotated to represent 2′-cysteinyldopa and 5′-cysteinyldopa, respectively (mean±SEM; n=3; *** P<0.001). FIG. 15D shows cystine levels in SKMEL30 whole-cell lysates and purified melanosomes (mean±SEM; n=3, *** P<0.001). FIG. 15E shows levels of cysteinyldopas in SKMEL30 cells (mean±SEM; n=4; *** P<0.001). FIG. 15F shows SKMEL30 cells lacking MFSD12 are darker than their wild-type counterparts. Photographs are of pellets containing 3 million SKMEL30 cells at the bottom of 1.5 mL tubes. FIG. 15G provides a schematic of proposed function for MFSD12 in melanosomes, whereby it controls the influx of cysteine into melanosomes, enabling the production of cystine, cysteinyldopas, and pheomelanin. FIGS. 15A-15G re-present data from FIGS. 1A-1B and FIGS. 4A-4B, as well as providing additional data.

FIGS. 16A-16F demonstrate MFSD12 is necessary to maintain lysosomal cystine and cysteine levels. FIG. 16A shows expression of pigmentation genes across tissues. FANTOM5 expression profiling data from Human Protein Atlas displayed as scaled tags per million (Kawaji et al., 2017; Uhlén et al., 2015). Full labels of tissues and additional pigmentation genes are displayed in FIG. 20A. FIG. 16B shows untargeted polar metabolite profiling of lysosomes isolated from MFSD12 knock-out and wild-type HEK-293T cells (n=6 per condition). FIG. 16C provides follow-up metabolite analysis of cystine and cysteine in whole-cell extracts and purified lysosomes. Samples were split and run via standard methods for cystine and derivatized with Ellman's reagent to enable cysteine detection (mean±SEM; n=3; * P<0.05, *** P<0.001). FIG. 16D shows cystine measurements in whole-cell extracts and purified lysosomes. To generate MFSD12&CTNS combined knock-out cells, MFSD12 was deleted from the CTNS knock-out cells (mean±SEM; n=3; **P<0.01, *** P<0.001). FIG. 16E shows cystine measurements in whole-cell extracts and purified lysosomes from patient-derived fibroblasts. Matched unaffected parental (GM00906) and cystinotic patient (GM00379) fibroblast cell lines were obtained from the Coriell institute. Cells were grown to confluence to arrest division before cystine was measured (mean±SEM; n=4-6; ** P<0.01, *** P<0.001). FIG. 16F shows a schematic of proposed role of MFSD12 in lysosomes in which it is upstream of CTNS in the lysosomal cystine/cysteine cycle. FIGS. 16A-16F re-present data from FIGS. 6-8, as well as providing additional data.

FIGS. 17A-17E show MFSD12 is necessary, and likely sufficient, for the import of cysteine into melanosomes and lysosomes. FIGS. 17A-17B show measurements of [¹⁴C] in melanosomes and lysosomes from cells exposed to [¹⁴C]-cystine in the media. [¹⁴C] carbons are indicated by red points in ball and stick models, as are expected chemical changes to cystine in different cellular compartments. MelanoTag-expressing B16F10 cells or LysoTag-expressing HEK-293T cells were incubated with 1.2 μM [¹⁴C] cystine in the media for 1 hour, upon which organelles were isolated via the MelanolP or LysolP methods. Background signal, determined from a sample treated with 1% Triton X-100 during the immunoprecipitation and wash-steps, was subtracted from the signal for the immunoprecipitation samples. For the lysosomal assays (FIG. 17B), whole-cell and immunoprecipate samples were normalized using a hexosaminidase assay (mean±SEM; n=3; ** P<0.01, *** P<0.001). FIG. 17C shows cysteine uptake assay into isolated lysosomes. Lysosomes were prepared by differential centrifugation and the recovered material normalized by the hexosaminidase assay. In the non-competed samples, 35 μM unlabeled cysteine was added with trace amounts of [³⁵S]-cysteine (mean±SEM; n=3, *** P<0.001). FIG. 17D shows immunofluorescence analysis of HEK-293T cells expressing wild-type MFDS12 or the MFSD12^{LL253-254AA} mutant (MFSD12^{PM}). Wheat-germ agglutinin (WGA) was used to mark the plasma membrane. The HA-MFSD12^{PM} protein was visualized with an anti-HA epitope tag antibody. FIG. 17E shows cysteine uptake in whole-cells expressing MFSD12^{PM}. Cells expressing the indicated proteins via transfection were incubated in a buffer containing inhibitors of native cysteine transport before the addition of cysteine. Under the non-competed conditions, 10 μM unlabeled cysteine was added with a trace amount of [³⁵S]-cysteine (mean±SEM; n=3, *** P<0.001).

FIG. 18 shows follow-up analysis with standard validated m/z and internal normalization of ‘proteogenic amino acids’ highlighted in untargeted metabolite profiling of wild-type and Tyr knock-out melanosomes (FIG. 14D). Amino acids are presented in order of increasing retention time (mean±S.E.M.; n=3; * P<0.05, *** P<0.001).

FIGS. 19A-19D show the loss of MFSD12 reduces the levels of cysteinyldopas. FIG. 19A shows cysteinyldopas were synthesized according to an adapted protocol from Ito and Prota, 1977 (Ito and Prota, 1977). Two species, distinguished by retention time, were generated at the expected m/z for cysteinyldopas. It has been shown 5′-cysteinyldopa is produced in greater abundance to 2′-cysteinyldopa in this reaction. Taking MS1 peak intensity to approximate abundance, the ‘Minor Isomer’ was putatively annotated as 2′ substituted, and the ‘Major Isomer’ as 5′ substituted. FIG. 19B shows a mirror plot of ddMS2 data comparing 2′- and 5′-cysteinyldopa in synthetic cysteinyldopas. FIGS. 19C-19D provide mirror plots of ddMS2 peaks displaying similarities in ddMS2 spectra of 2′- and 5′-cysteinyldopa species in biological samples (B16F10 extracts) and synthetic standards.

FIGS. 20A-20C demonstrate MFSD12 is necessary to maintain lysosomal cystine and cysteine levels. FIG. 20A shows FANTOM5 CAGE profiling data accessed via Human Protein Atlas (Kawaji et al., 2017; Uhlén et al., 2015). Six representational pigmentation genes, including MFSD12, are shown. FIG. 20B shows metabolite profiling of LysolP samples from HEK-293T cells comparing lysosomes from wild-type and MFSD12 knock-out cells (mean±S.E.M.; n=4; *P<0.05, ***P<0.001).

FIG. 20C shows lentiviral shRNA knock-down of MFSD12 quantified via qPCR and normalized to ACTB levels (mean±S.E.M., n=3, *** P<0.001). FIGS. 20A-20C re-present data from FIGS. 16A-16F, as well as providing additional data.

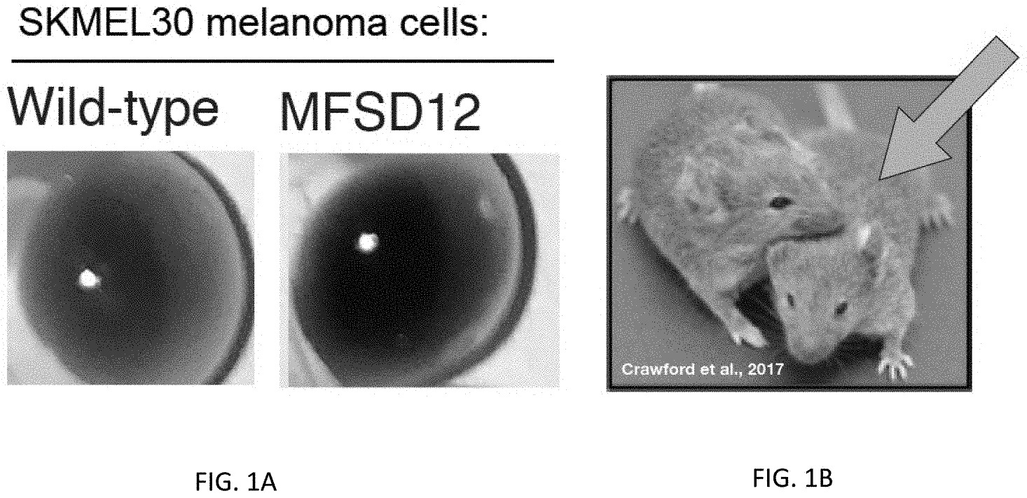

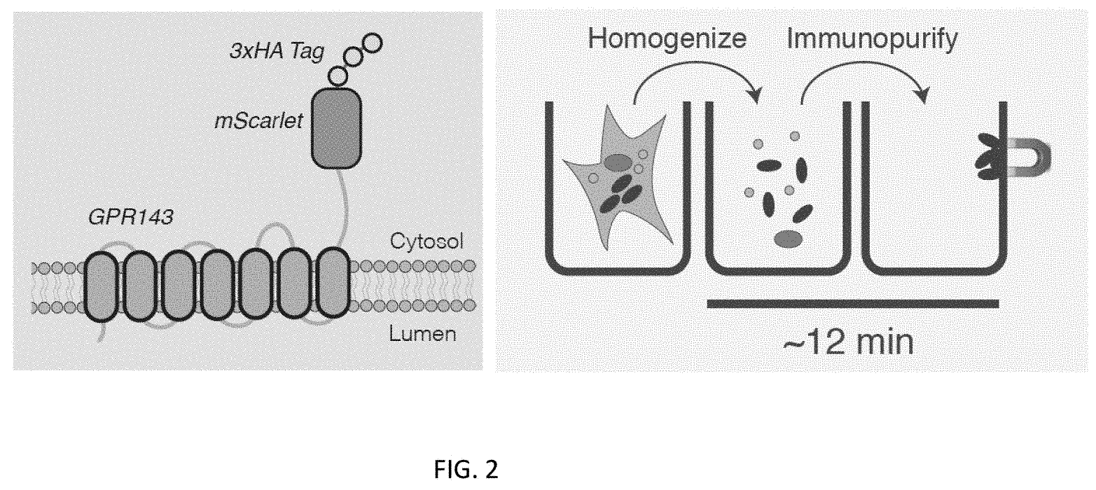

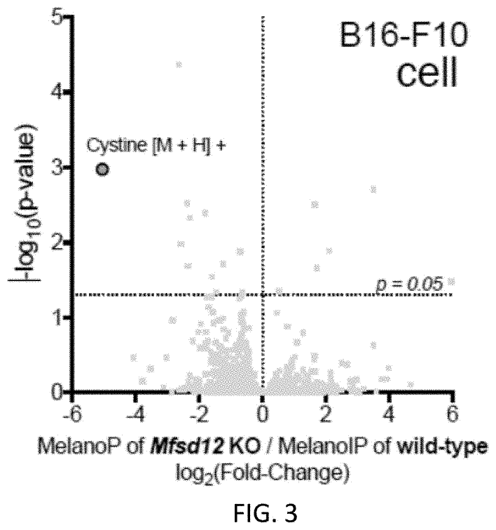

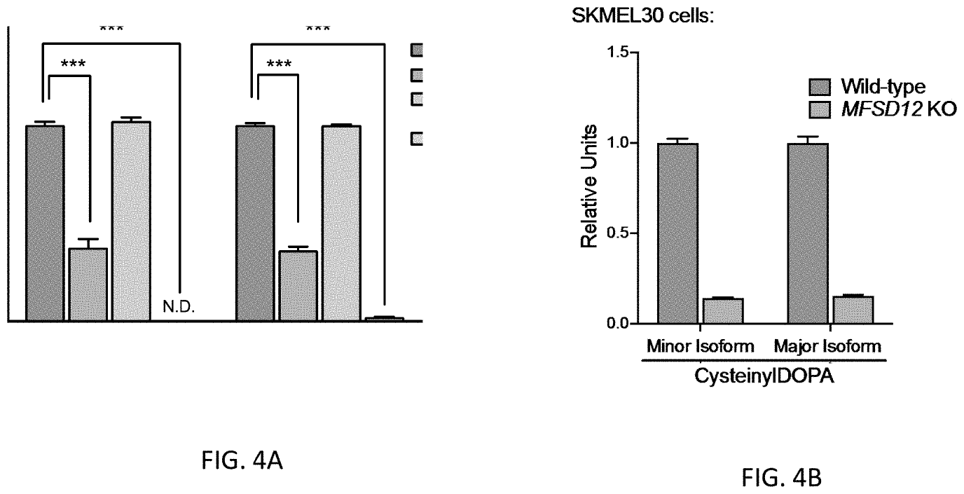

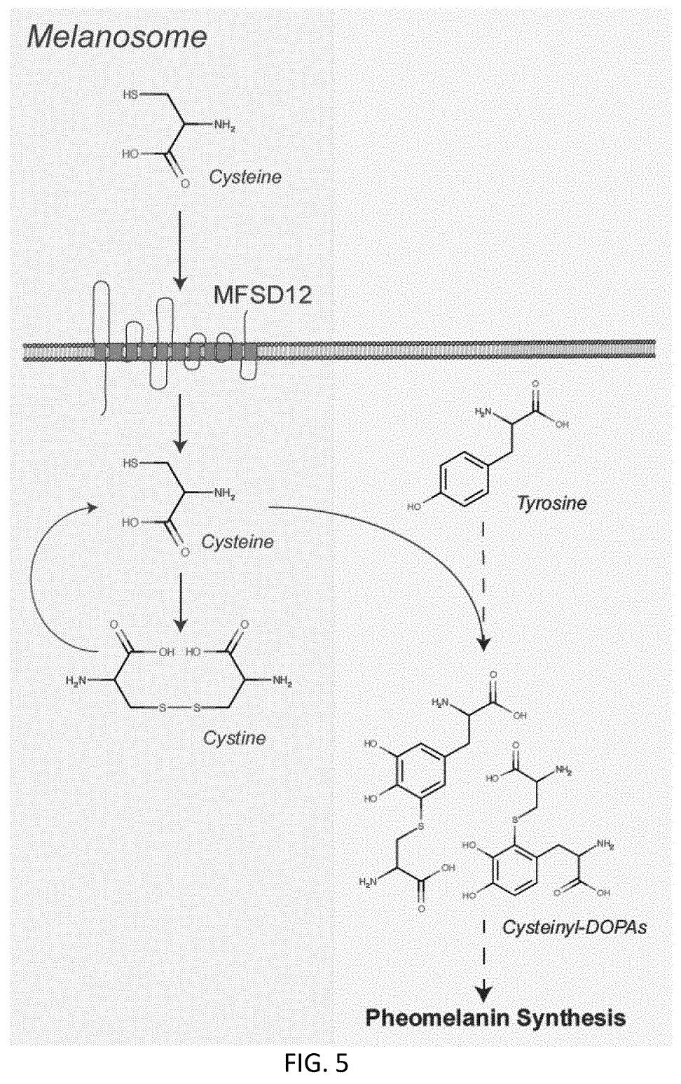

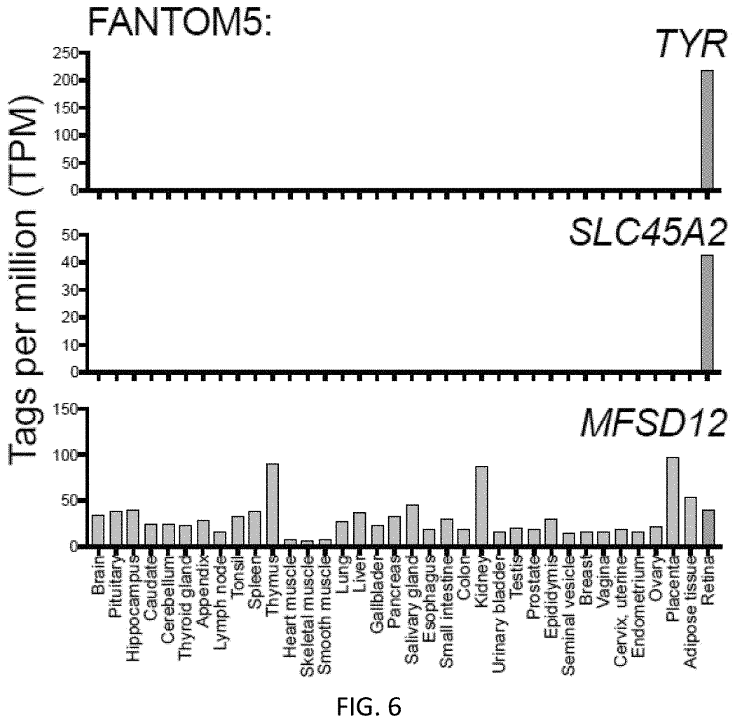

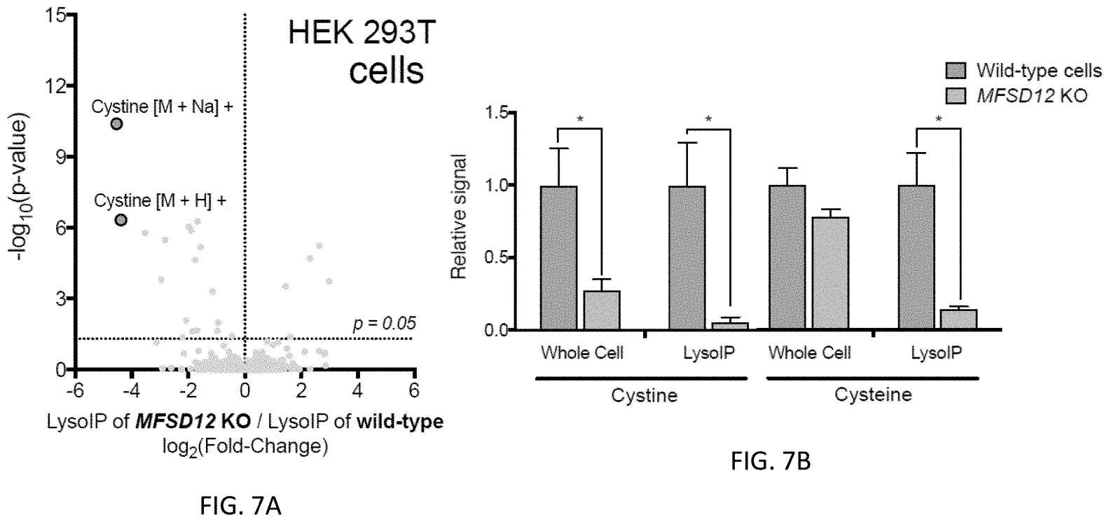

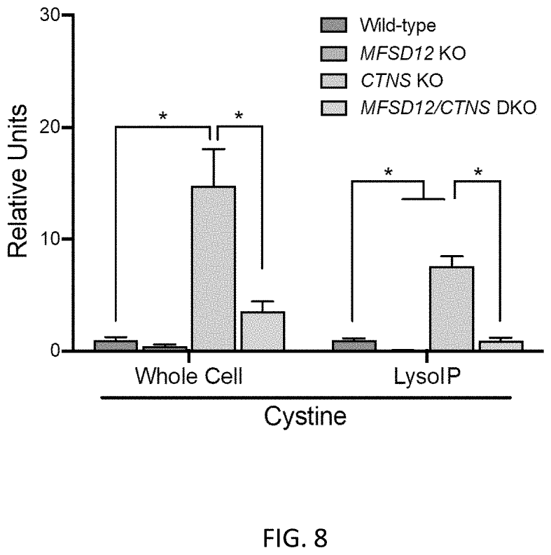

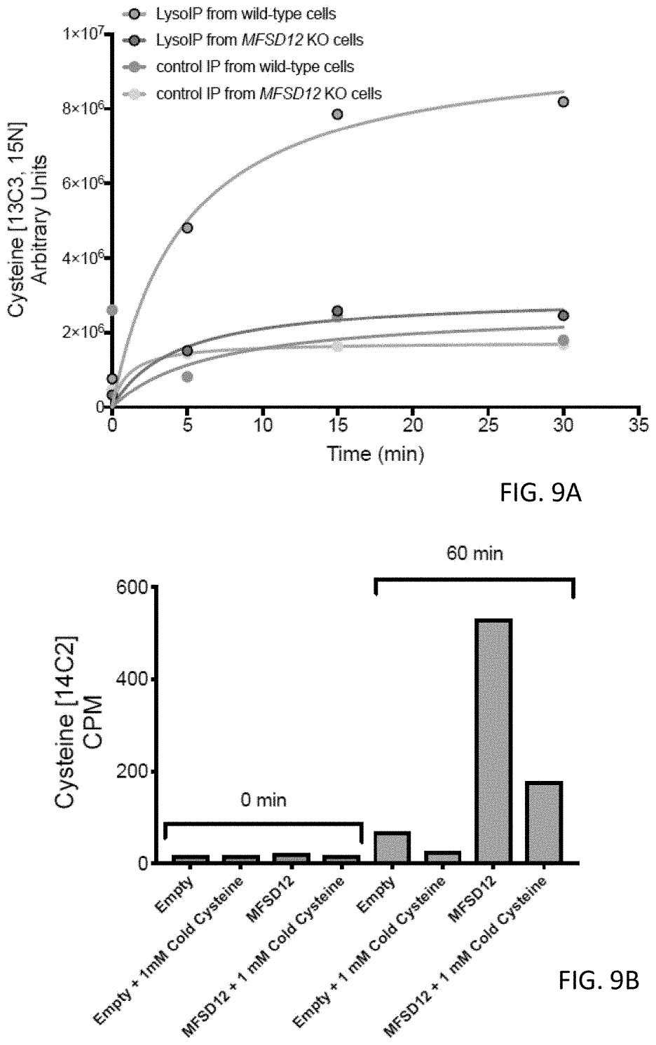

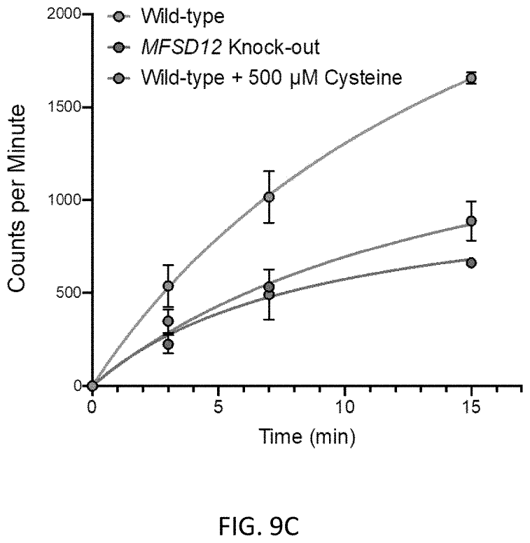

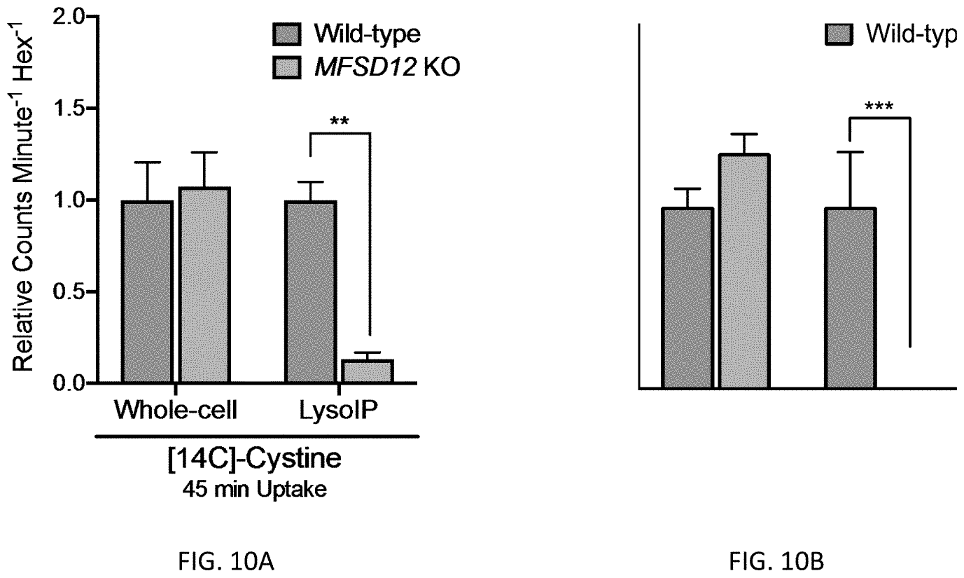

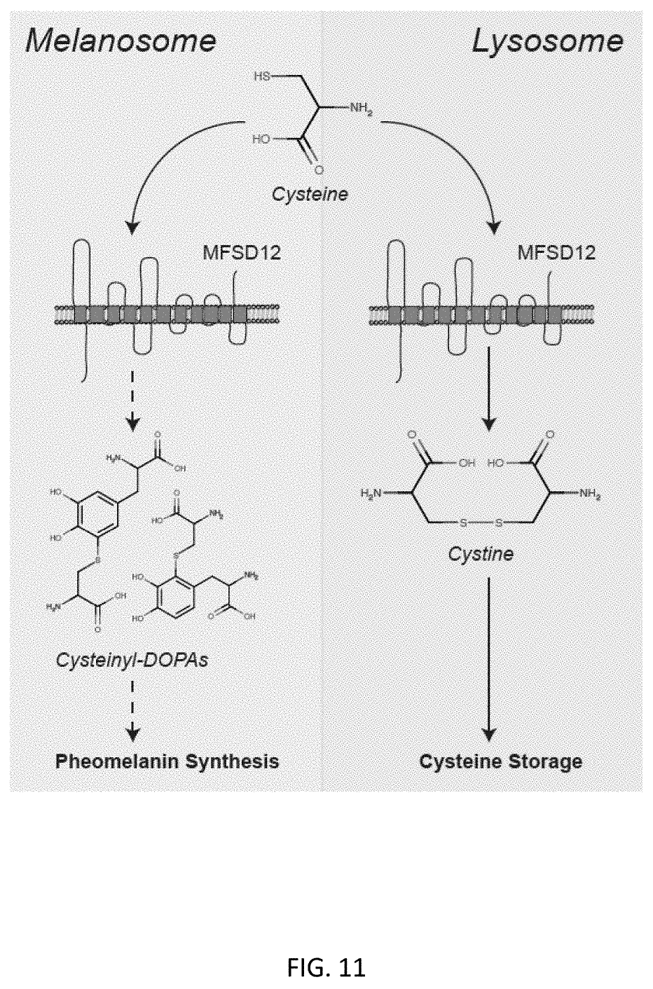

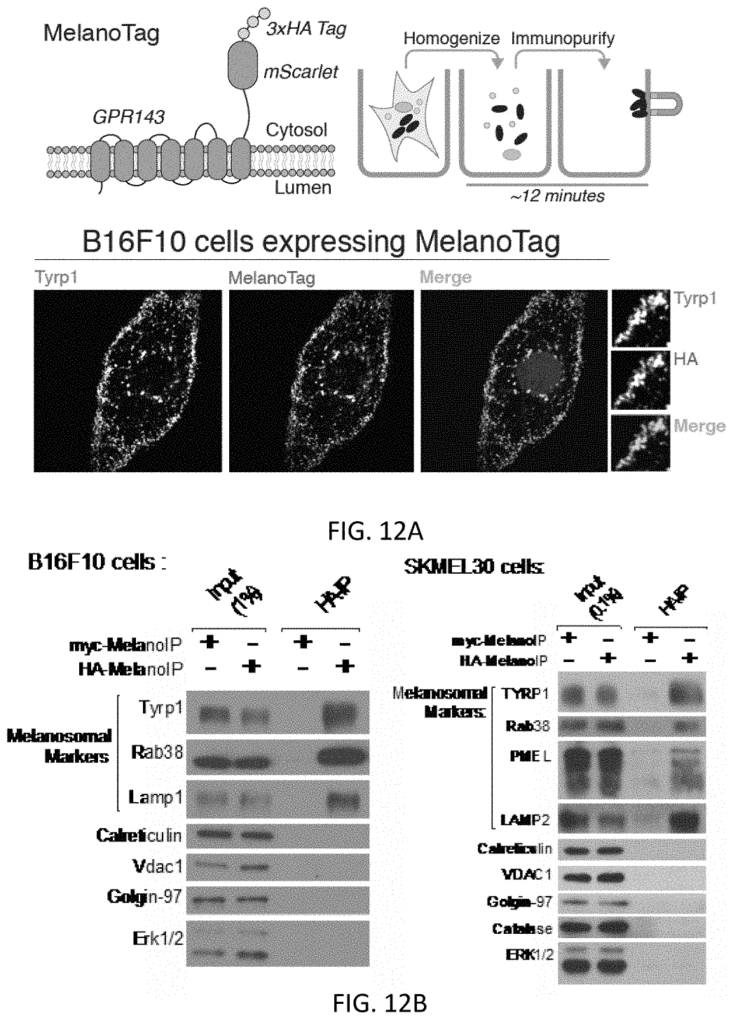

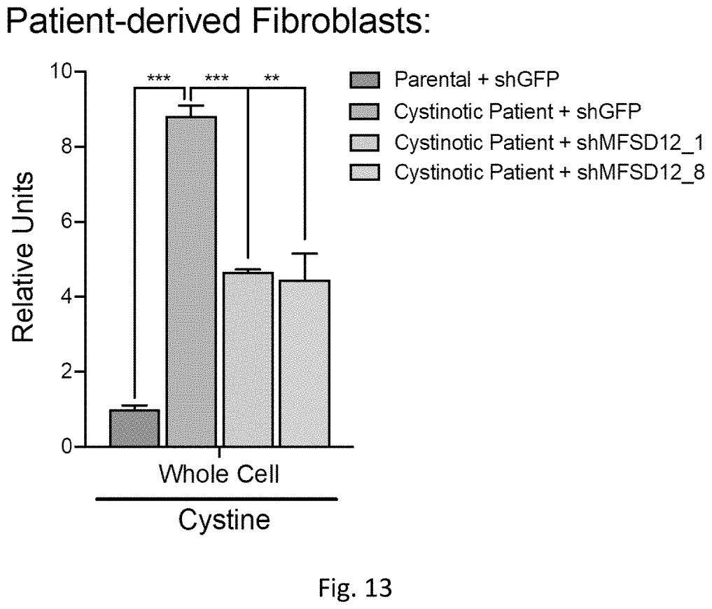

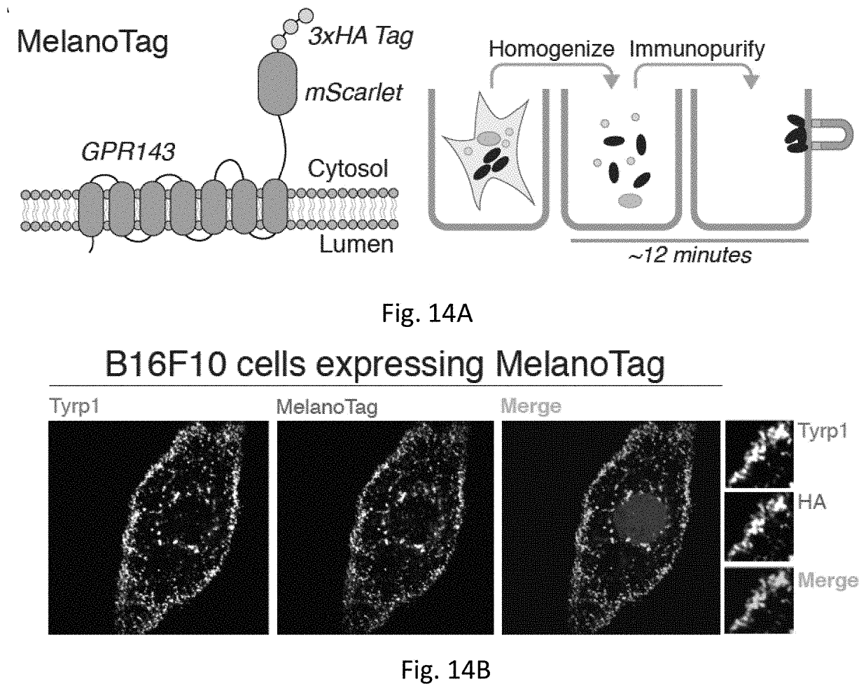

### DETAILED DESCRIPTION OF THE INVENTION

Dozens of genes contribute to the vast variation in human pigmentation. Many of these encode proteins that localize to the melanosome, the lysosome-related organelle that synthesizes pigment, but have unclear functions. Described herein is the MelanolP method used for rapidly isolating melanosomes and profiling their labile metabolite contents. This method is used to study MFSD12, a transmembrane protein of unknown molecular function that when suppressed causes darker pigmentation in mice and humans. It was found that MFSD12 is required to maintain normal levels of cystine, the oxidized dimer of cysteine, in melanosomes, and to produce cysteinyldopas, the precursors of pheomelanin synthesis made in melanosomes via cysteine oxidation. Tracing and biochemical analyses show that MFSD12 is necessary for the import of cysteine into melanosomes, and, in non-pigmented cells, lysosomes. Indeed, loss of MFSD12 reduced the accumulation of cystine in lysosomes of fibroblasts from patients with cystinosis, a lysosomal storage disease caused by inactivation of the lysosomal cystine exporter CTNS (Cystinosin). Thus, MFSD12 is an essential component of the long-sought cysteine importer for melanosomes and lysosomes.

Disclosed herein are therapeutic strategies that have overlapping uses of treating rare diseases, as well as providing cosmetic benefits. Modulating the expression of major facilitator superfamily domain containing 12 (MFSD12) is shown to be beneficial for modulating (e.g., decreasing) or stabilizing cysteine transport function in a cell (e.g., in the lysosome and/or the melanosome of the cell). In some embodiments, decreasing or stabilizing cysteine transport in a cell is effective for treating or preventing a lysosomal storage disease or disorder, and/or for providing skin darkening effects. Also disclosed herein are compositions for modulating the expression of MFSD12 and/or the activity of a gene product of MFSD12, as well as assays for identifying agents that may act as modulators of MFSD12. MFSD12 (NCBI Gene ID 126321; OMIM 617745; Gen Bank BC036706.2), is a member of the major facilitator superfamily (MFS). The MFS is a superfamily of membrane transport proteins that facilitate movement of small molecules across cell membranes.

Methods of Modulating or Stabilizing Cysteine Transport

Aspects of the disclosure are directed to methods of modulating or stabilizing cysteine transport function in a lysosome and/or in a melanosome of a cell (e.g., modulating or stabilizing the import of cysteine into a lysosome and/or a melanosome). In some embodiments, the modulation or stabilization of cysteine transport is in the lysosome of the cell. In some embodiments, the modulation or stabilization of cysteine transport is in the melanosome of the cell. In some embodiments, cysteine transport function in a lysosome and/or in a melanosome of a cell is restored to wild-type levels. It is generally understood that upon transport into the lysosome (e.g., by MFSD12) cysteine is converted by oxidation to the dimeric form cystine, which can readily be reduced back to cysteine in the cytosol or, presumably, if it is removed from the lysosome. Reference to cysteine herein encompasses cysteine in the form of cystine.

In some embodiments, cysteine transport function is modulated or stabilized by modulating the expression of MFSD12 or the activity of a gene product of MFSD12 (e.g., in the cell). As used herein “modulating” or “modulates” means causing or facilitating a qualitative or quantitative change, alteration, or modification. Without limitation, such change may be an increase or decrease in a qualitative or quantitative aspect. For example, modulating transcription of a gene includes increasing or decreasing the rate or frequency of gene transcription. As used herein, “stabilizing” or “stabilizes” means the rate of increase or decrease of a certain level is reduced or stopped, and fluctuations in certain levels are reduced or eliminated.

In some embodiments, the level of cysteine in the lysosome and/or in the melanosome is increased. In some embodiments, the expression of MFSD12 or the activity of a gene product of MFSD12 is increased, thereby increasing the level of cysteine in the lysosome and/or in the melanosome. In some embodiments, the level of cysteine in the lysosome and/or in the melanosome is decreased. In some embodiments, the level of cysteinyldopa in the lysosome and/or in the melanosome is decreased. In some embodiments, the level of cysteine in the lysosome and/or in the melanosome is decreased by about 50%-95%. In some embodiments, the level of cysteine in the lysosome and/or in the melanosome is decreased by about 60%-90%, 65%-85%, or 70%-80%. In some embodiments, the level of cysteine in the lysosome and/or in the melanosome is decreased by about 50%-60%, 60%-70%, or 55%-90%. In some embodiments, the level of cysteine in the lysosome and/or in the melanosome is decreased by about 75% to 95% or 80% to 90%. In some embodiments, the level of cysteine in the lysosome and/or in the melanosome is decreased by about 95%. In some embodiments, the level of cysteine in the lysosome and/or in the melanosome is decreased by about 10-fold, 15-fold, 20-fold, 25-fold, 30-fold, 35-fold, 40-fold, 45-fold, or 50-fold. In some embodiments, the expression of MFSD12 or the activity of a gene product of MFSD12 is decreased, thereby decreasing the level of cysteine in the lysosome and/or in the melanosome. In some embodiments, the level of cysteine in the lysosome and/or in the melanosome is stabilized.

In some embodiments, the expression of MFSD12 or the activity of a gene product of MFSD12 is increased by at least about 5%, 10%, 15%, 20%, 25%, 30%, 35%, 40%, 45%, 50%, 55%, 60%, 65%, 70%, 75%, 80%, 85%, 90%, 95%, 99% or more. In some embodiments, the expression of MFSD12 or the activity of a gene product of MFSD12 is increased by at least about 1.1 fold, at least 1.2 fold, 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 20 fold, at least 30 fold, at least 40 fold, at least 50 fold, or at least 100 fold, at least a 1,000 fold, at least 10,000 fold as compared to MFSD12 that has not been modulated. In some embodiments, the expression of MFSD12 or the activity of a gene product of MFSD12 is reduced by at least about 5%, 10%, 15%, 20%, 25%, 30%, 35%, 40%, 45%, 50%, 55%, 60%, 65%, 70%, 75%, 80%, 85%, 90%, 95%, 99% or more.

In some embodiments, the expression of MFSD12 or the activity of a gene product of MFSD12 is modulated by contacting the cell with an agent and/or utilizing any known gene editing technique (e.g., CRISPR, TALEN, ZFN, etc.). “Contacting”, “contacting a cell” and similar terms as used herein, refer to any means of introducing an agent (e.g., a nucleic acid, peptide, antibody, small molecule, etc.) into a target cell, including chemical and physical means, whether directly or indirectly or whether the agent physically contacts the cell directly or is introduced into an environment in which the cell is present. Contacting is intended to encompass methods of exposing a cell, delivering to a cell, or “loading” a cell with an agent by viral or non-viral vectors, wherein such agent is bioactive upon delivery or wherein such agent is processed intracellularly to an active form. The method of delivery will be chosen for the particular agent and use (e.g., disease being treated). Parameters that affect delivery, as is known in the medical art, can include, inter alia, the cell type affected, and cellular location. In some embodiments, contacting includes administering the agent to a subject.

In some embodiments, the expression of MFSD12 or the activity of a gene product of MFSD12 is modulated by contacting the cell with an agent (e.g., an effective amount of an agent). “Agent” is used herein to broadly refer to any substance, compound (e.g., molecule), supramolecular complex, material, or combination or mixture thereof. In some aspects, an agent can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, peptide mimetics, analogs, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

An “analog” of a first agent refers to a second agent that is structurally and/or functionally similar to the first agent. A “structural analog” of a first agent is an analog that is structurally similar to the first agent. Unless otherwise specified, the term “analog” as used herein refers to a structural analog. A structural analog of an agent may have substantially similar physical, chemical, biological, and/or pharmacological propert(ies) as the agent or may differ in at least one physical, chemical, biological, or pharmacological property. In some embodiments at least one such property differs in a manner that renders the analog more suitable for a purpose of interest. In some embodiments a structural analog of an agent differs from the agent in that at least one atom, functional group, or substructure of the agent is replaced by a different atom, functional group, or substructure in the analog. In some embodiments, a structural analog of an agent differs from the agent in that at least one hydrogen or substituent present in the agent is replaced by a different moiety (e.g., a different substituent) in the analog.

In some embodiments, the agent is a nucleic acid. The term “nucleic acid” refers to polynucleotides such as deoxyribonucleic acid (DNA) and ribonucleic acid (RNA). The terms “nucleic acid” and “polynucleotide” are used interchangeably herein and should be understood to include double-stranded polynucleotides, single-stranded (such as sense or antisense) polynucleotides, and partially double-stranded polynucleotides. A nucleic acid often comprises standard nucleotides typically found in naturally occurring DNA or RNA (which can include modifications such as methylated nucleobases), joined by phosphodiester bonds. In some embodiments a nucleic acid may comprise one or more non-standard nucleotides, which may be naturally occurring or non-naturally occurring (i.e., artificial; not found in nature) in various embodiments and/or may contain a modified sugar or modified backbone linkage. Nucleic acid modifications (e.g., base, sugar, and/or backbone modifications), non-standard nucleotides or nucleosides, etc., such as those known in the art as being useful in the context of RNA interference (RNAi), aptamer, CRISPR technology, polypeptide production, reprogramming, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the translation, potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications. Where the length of a nucleic acid or nucleic acid region is given in terms of a number of nucleotides (nt) it should be understood that the number refers to the number of nucleotides in a single-stranded nucleic acid or in each strand of a double-stranded nucleic acid unless otherwise indicated. An “oligonucleotide” is a relatively short nucleic acid, typically between about 5 and about 100 nt long. In some embodiments, the nucleic acid codes for MFSD12 or functional variants thereof.

The term “RNA interference” (RNAi) encompasses processes in which a molecular complex known as an RNA-induced silencing complex (RISC) reduces gene expression in a sequence-specific manner in, e.g., eukaryotic cells, e.g., vertebrate cells, or in an appropriate in vitro system. RISC may incorporate a short nucleic acid strand (e.g., about 16-about 30 nucleotides (nt) in length) that pairs with and directs or “guides” sequence-specific degradation or translational repression of RNA (e.g., mRNA) to which the strand has complementarity. The short nucleic acid strand may be referred to as a “guide strand” or “antisense strand”. An RNA strand to which the guide strand has complementarity may be referred to as a “target RNA”. A guide strand may initially become associated with RISC components (in a complex sometimes termed the RISC loading complex) as part of a short double-stranded RNA (dsRNA), e.g., a short interfering RNA (siRNA). The other strand of the short dsRNA may be referred to as a “passenger strand” or “sense strand”. The complementarity of the structure formed by hybridization of a target RNA and the guide strand may be such that the strand can (i) guide cleavage of the target RNA in the RNA-induced silencing complex (RISC) and/or (ii) cause translational repression of the target RNA. Reduction of expression due to RNAi may be essentially complete (e.g., the amount of a gene product is reduced to background levels) or may be less than complete in various embodiments. For example, mRNA and/or protein level may be reduced by 50%, 60%, 70%, 75%, 80%, 85%, 90%, 95%, or more, in various embodiments. As known in the art, the complementarity between the guide strand and a target RNA need not be perfect (100%) but need only be sufficient to result in inhibition of gene expression. For example, in some embodiments 1, 2, 3, 4, 5, or more nucleotides of a guide strand may not be matched to a target RNA. “Not matched” or “unmatched” refers to a nucleotide that is mismatched (not complementary to the nucleotide located opposite it in a duplex, i.e., wherein Watson-Crick base pairing does not take place) or forms at least part of a bulge. Examples of mismatches include, without limitation, an A opposite a G or A, a C opposite an A or C, a U opposite a C or U, a G opposite a G. A bulge refers to a sequence of one or more nucleotides in a strand within a generally duplex region that are not located opposite to nucleotide(s) in the other strand. “Partly complementary” refers to less than perfect complementarity. In some embodiments a guide strand has at least about 80%, 85%, or 90%, e.g., at least about 91%, 92%, 93%, 94%, 95%, 96%, 97%, 98%, 99%, or 100% sequence complementarity to a target RNA over a continuous stretch of at least about 15 nt, e.g., between 15 nt and 30 nt, between 17 nt and 29 nt, between 18 nt and 25 nt, between 19 nt and 23 nt, of the target RNA. In some embodiments at least the seed region of a guide strand (the nucleotides in positions 2-7 or 2-8 of the guide strand) is perfectly complementary to a target RNA. In some embodiments, a guide strand and a target RNA sequence may form a duplex that contains no more than 1, 2, 3, or 4 mismatched or bulging nucleotides over a continuous stretch of at least 10 nt, e.g., between 10-30 nt. In some embodiments a guide strand and a target RNA sequence may form a duplex that contains no more than 1, 2, 3, 4, 5, or 6 mismatched or bulging nucleotides over a continuous stretch of at least 12 nt, e.g., between 10-30 nt. In some embodiments, a guide strand and a target RNA sequence may form a duplex that contains no more than 1, 2, 3, 4, 5, 6, 7, or 8 mismatched or bulging nts over a continuous stretch of at least 15 nt, e.g., between 10-30 nt. In some embodiments, a guide strand and a target RNA sequence may form a duplex that contains no mismatched or bulging nucleotides over a continuous stretch of at least 10 nt, e.g., between 10-30 nt. In some embodiments, between 10-30 nt is 10, 11, 12, 13, 14, 15, 16, 17, 18, 19, 20, 21, 22, 23, 24, 25, 26, 27, 28, 29, or 30 nt.

As used herein, the term “RNAi agent” encompasses nucleic acids that can be used to achieve RNAi in eukaryotic cells. Short interfering RNA (siRNA), short hairpin RNA (shRNA), and microRNA (miRNA) are examples of RNAi agents. siRNAs typically comprise two separate nucleic acid strands that are hybridized to each other to form a structure that contains a double stranded (duplex) portion at least 15 nt in length, e.g., about 15-about 30 nt long, e.g., between 17-27 nt long, e.g., between 18-25 nt long, e.g., between 19-23 nt long, e.g., 15, 16, 17, 18, 19, 20, 21, 22, 23, 24, 25, 26, 27, 28, 29, or 30 nucleotides. In some embodiments the strands of an siRNA are perfectly complementary to each other within the duplex portion. In some embodiments the duplex portion may contain one or more unmatched nucleotides, e.g., one or more mismatched (non-complementary) nucleotide pairs or bulged nucleotides. In some embodiments either or both strands of an siRNA may contain up to about 1, 2, 3, or 4 unmatched nucleotides within the duplex portion. In some embodiments a strand may have a length of between 15-35 nt, e.g., between 17-29 nt, e.g., 19-25 nt, e.g., 21-23 nt. Strands may be equal in length or may have different lengths in various embodiments. In some embodiments strands may differ by 1-10 nt in length. A strand may have a 5′ phosphate group and/or a 3′ hydroxyl (—OH) group. Either or both strands of an siRNA may comprise a 3′ overhang of, e.g., about 1-10 nt (e.g., 1-5 nt, e.g., 2 nt). Overhangs may be the same length or different in lengths in various embodiments. In some embodiments an overhang may comprise or consist of deoxyribonucleotides, ribonucleotides, or modified nucleotides or modified ribonucleotides such as 2′-O-methylated nucleotides, or 2′-O-methyl-uridine. An overhang may be perfectly complementary, partly complementary, or not complementary to a target RNA in a hybrid formed by the guide strand and the target RNA in various embodiments.

shRNAs are nucleic acid molecules that comprise a stem-loop structure and a length typically between about 40-150 nt, e.g., about 50-100 nt, e.g., about 60-80 nt. A “stem-loop structure” (also referred to as a “hairpin” structure) refers to a nucleic acid having a secondary structure that includes a region of nucleotides which are known or predicted to form a double strand (stem portion; duplex) that is linked on one side by a region of (usually) predominantly single-stranded nucleotides (loop portion). Such structures are well known in the art and the term is used consistently with its meaning in the art. A guide strand sequence may be positioned in either arm of the stem, i.e., 5′ with respect to the loop or 3′ with respect to the loop in various embodiments. As is known in the art, the stem structure does not require exact base-pairing (perfect complementarity). Thus, the stem may include one or more unmatched residues or the base-pairing may be exact, i.e., it may not include any mismatches or bulges. In some embodiments the stem is between 15-30 nt, e.g., between 17-29 nt, e.g., between 19-25 nt. In some embodiments the stem is between 15-19 nt. In some embodiments the stem is between 19-30 nt. The primary sequence and number of nucleotides within the loop may vary. Examples of loop sequences include, e.g., UGGU; ACUCGAGA; UUCAAGAGA. In some embodiments a loop sequence found in a naturally occurring miRNA precursor molecule (e.g., a pre-miRNA) may be used. In some embodiments a loop sequence may be absent (in which case the termini of the duplex portion may be directly linked). In some embodiments a loop sequence may be at least partly self-complementary. In some embodiments the loop is between 1 and 20 nt in length, e.g., 1-15 nt, e.g., 4-9 nt. The shRNA structure may comprise a 5′ or 3′ overhang. As known in the art, an shRNA may undergo intracellular processing, e.g., by the ribonuclease (RNase) III family enzyme known as Dicer, to remove the loop and generate an siRNA.

Mature endogenous miRNAs are short (typically 18-24 nt, e.g., about 22 nt), single-stranded RNAs that are generated by intracellular processing from larger, endogenously encoded precursor RNA molecules termed miRNA precursors (see, e.g., Bartel, D., Cell. 116(2):281-97 (2004); Bartel D P. Cell. 136(2):215-33 (2009); Winter, J., et al., Nature Cell Biology 11: 228-234 (2009). Artificial miRNA may be designed to take advantage of the endogenous RNAi pathway in order to silence a target RNA of interest. The sequence of such artificial miRNA may be selected so that one or more bulges is present when the artificial miRNA is hybridized to its target sequence, mimicking the structure of naturally occurring miRNA:mRNA hybrids. Those of ordinary skill in the art are aware of how to design artificial miRNA.

An RNAi agent that contains a strand sufficiently complementary to an RNA of interest so as to result in reduced expression of the RNA of interest (e.g., as a result of degradation or repression of translation of the RNA) in a cell or in an in vitro system capable of mediating RNAi and/or that comprises a sequence that is at least 80%, 90%, 95%, or more (e.g., 100%) complementary to a sequence comprising at least 10, 12, 15, 17, or 19 consecutive nucleotides of an RNA of interest may be referred to as being “targeted to” the RNA of interest. An RNAi agent targeted to an RNA transcript may also be considered to be targeted to a gene from which the transcript is transcribed.

In some embodiments an RNAi agent is a vector (e.g., an expression vector) suitable for causing intracellular expression of one or more transcripts that give rise to a siRNA, shRNA, or miRNA in the cell. Such a vector may be referred to as an “RNAi vector”. An RNAi vector may comprise a template that, when transcribed, yields transcripts that may form a siRNA (e.g., as two separate strands that hybridize to each other), shRNA, or miRNA precursor (e.g., pri-miRNA or pre-mRNA).

An RNAi agent may be produced in any of a variety of ways in various embodiments. For example, nucleic acid strands may be chemically synthesized (e.g., using standard nucleic acid synthesis techniques) or may be produced in cells or using an in vitro transcription system. Strands may be allowed to hybridize (anneal) in an appropriate liquid composition (sometimes termed an “annealing buffer”). An RNAi vector may be produced using standard recombinant nucleic acid techniques.

In some embodiments, the agent is a small molecule. The term “small molecule” refers to an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

In some embodiments, the agent is a protein or polypeptide. The term “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, a small molecule (such as a fluorophore), etc.

In some embodiments, the agent is a peptide mimetic. The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics.

In some embodiments, the agent is encoded by a synthetic RNA (e.g., modified mRNAs). The synthetic RNA can encode any suitable agent described herein. Synthetic RNAs, including modified RNAs are taught in WO 2017075406, which is herein incorporated by reference. In some embodiments, the agent is a synthetic RNA.

In some embodiments, the expression of MFSD12 or the activity of a gene product of MFSD12 is modulated utilizing any known gene editing technique (e.g., CRISPR, TALEN, ZFN, etc.). In some embodiments, the expression of MFSD12 or the activity of a gene product of MFSD12 is modulated utilizing CRISPR.

In some embodiments, a catalytically inactive site specific nuclease and an effector domain capable of attaching a DNA, RNA, or protein to the nucleotide sequence is used as the agent. In some embodiments, the catalytically inactive site specific nuclease dCas (e.g., dCas9 or Cpf1) is used as the agent. The agent may reduce or increase expression of MFSD12 (e.g., via modulating methylation of genomic DNA involved in expression of MFSD12) or reduce or increase activity of MFSD12 (e.g., by modifying the coding sequence for MFSD12). In some embodiments, the agent is a dCas-transcription activator domain fusion protein that enhances transcription of MFSD12 in the presence of the appropriate guide sequence.

A variety of CRISPR associated (Cas) genes or proteins which are known in the art can be modified to make a catalytically inactive site specific nuclease, the choice of Cas protein will depend upon the particular conditions of the method (e.g., ncbi.nlm.nih.govigene/?term=cas9). Specific examples of Cas proteins include Cas1, Cas2, Cas3, Cas4, Cas5, Cas6, Cas7, Cas8, Cas9 and Cas10. In a particular aspect, the Cas nucleic acid or protein used in the methods is Cas9. In some embodiments a Cas protein, e.g., a Cas9 protein, may be from any of a variety of prokaryotic species. In some embodiments a particular Cas protein, e.g., a particular Cas9 protein, may be selected to recognize a particular protospacer-adjacent motif (PAM) sequence. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Cryptococcus, a Corynebacterium, a Haemophilus, a Eubacterium, a Pasteurella, a Prevotella, a VeiUonella, or a Marinobacter. In some embodiments nucleic acids encoding two or more different Cas proteins, or two or more Cas proteins, may be introduced into a cell, zygote, embryo, or animal, e.g., to allow for recognition and modification of sites comprising the same, similar or different PAM motifs.

In some embodiments, the Cas protein is Cpf1 protein or a functional portion thereof. In some embodiments, the Cas protein is Cpf1 from any bacterial species or functional portion thereof. In certain embodiments, a Cpf1 protein is a Francisella novicida U112 protein or a functional portion thereof, an Acidaminococcus sp. BV3L6 protein or a functional portion thereof, or a Lachnospiraceae bacterium ND2006 protein or a function portion thereof. Cpf1 protein is a member of the type V CRISPR systems. Cpf1 protein is a polypeptide comprising about 1300 amino acids. Cpf1 contains a RuvC-like endonuclease domain.

In some embodiments, a Cas9 nickase may be generated by inactivating one or more of the Cas9 nuclease domains. In some embodiments, an amino acid substitution at residue 10 in the RuvC I domain of Cas9 converts the nuclease into a DNA nickase. For example, the aspartate at amino acid residue 10 can be substituted for alanine (Cong et al, Science, 339:819-823). Other amino acids mutations that create a catalytically inactive Cas9 protein include mutating at residue 10 and/or residue 840. Mutations at both residue 10 and residue 840 can create a catalytically inactive Cas9 protein, sometimes referred to herein as dCas9. For example, a D10A and a H840A Cas9 mutant is catalytically inactive.

As used herein an “effector domain” is a molecule (e.g., protein) that modulates the expression and/or activation of a genomic sequence (e.g., gene). The effector domain may have methylation activity or demethylation activity (e.g., DNA methylation or DNA demethylation activity). In some aspects, the effector domain targets one or both alleles of a gene. The effector domain can be introduced as a nucleic acid sequence and/or as a protein. In some aspects, the effector domain can be a constitutive or an inducible effector domain. In some aspects, a Cas (e.g., dCas) nucleic acid sequence or variant thereof and an effector domain nucleic acid sequence are introduced into a cell. In some aspects, the effector domain is fused to a molecule that associates with (e.g., binds to) Cas protein (e.g., the effector molecule is fused to an antibody or antigen binding fragment thereof that binds to Cas protein). In some aspects, a Cas (e.g., dCas) protein or variant thereof and an effector domain are fused or tethered creating a chimeric protein and are introduced into the cell as the chimeric protein. In some aspects, the Cas (e.g., dCas) protein and effector domain bind as a protein-protein interaction. In some aspects, the Cas (e.g., dCas) protein and effector domain are covalently linked. In some aspects, the effector domain associates non-covalently with the Cas (e.g., dCas) protein. In some aspects, a Cas (e.g., dCas) nucleic acid sequence and an effector domain nucleic acid sequence are introduced as separate sequences and/or proteins. In some aspects, the Cas (e.g., dCas) protein and effector domain are not fused or tethered.

In some embodiments, the catalytically inactive site specific nuclease can be guided to specific DNA sites by one or more RNA sequences (sgRNA) to modulate activity and/or expression of one or more genomic sequences (e.g., exert certain effects on transcription or chromatin organization, or bring specific kind of molecules into specific DNA loci, or act as sensor of local histone or DNA state). In specific aspects, fusions of a dCas9 tethered with all or a portion of an effector domain create chimeric proteins that can be guided to specific DNA sites by one or more RNA sequences to modulate or modify methylation or demethylation of one or more genomic sequences. As used herein, a “biologically active portion of an effector domain” is a portion that maintains the function (e.g. completely, partially, minimally) of an effector domain (e.g., a “minimal” or “core” domain). The fusion of the Cas9 (e.g., dCas9) with all or a portion of one or more effector domains created a chimeric protein.

Examples of effector domains include a transcription activation domain (e.g., Gal4, Oaf1, Leu3, Rtg3, Pho4, Gln3, Gcn4, p53, NFAT, NF-κB, or VP16 transcription activation domain), chromatin organizer domain, a remodeler domain, a histone modifier domain, a DNA modification domain, a RNA binding domain, a protein interaction input devices domain (Grunberg and Serrano, Nucleic Acids Research, 3 ′8 (8): ′2663-267 ′5 (2010)), and a protein interaction output device domain (Grunberg and Serrano, Nucleic Acids Research, 3 ′8 (8): ′2663-267 ′5 (2010)). In some aspects, the effector domain is a DNA modifier. Specific examples of DNA modifiers include 5hmc conversion from 5mC such as Tet1 (Tet1CD); DNA demethylation by Tet1, ACID A, MBD4, Apobec1, Apobec2, Apobec3, Tdg, Gadd45a, Gadd45b, ROS1; DNA methylation by Dnmt1, Dnmt3a, Dnmt3b, CpG Methyltransferase M.SssI, and/or M.EcoHK31I. In specific aspects, an effector domain is Tet1. In other specific aspects, as effector domain is Dmnt3a. In some embodiments, dCas9 is fused to Tet1. In other embodiments, dCas9 is fused to Dnmt3a. Other examples of effector domains are described in PCT Application No. PCT/US2014/034387 and U.S. application Ser. No. 14/785,031, which are incorporated herein by reference in their entirety. Methods of using catalytically inactive site specific nuclease, effector domains for modifying a nucleotide sequence (e.g., genomic sequence), and sgRNA are taught in PCT/US2017/065918 filed 12 Dec. 2017, which is incorporated herein by reference.

Methods of Treatment

Some aspects of the disclosure are related to methods of treating or preventing a disease, disorder or dysfunction associated with aberrant cysteine transport function. As used herein, “cysteine transport function” refers to the ability of a cell or organelle to transport cysteine. In some aspects if cysteine transport is not functional or is ineffective, then cysteine may build up in the lysosome or the melanosome of a cell, or alternatively not enough cysteine may be present in the lysosome or the melanosome of a cell.

Some aspects of the disclosure are related to methods of treating or preventing a disease, disorder or dysfunction associated with an aberrant level of cysteine in a lysosome in a cell of a subject. In some embodiments the methods comprise, administering to the subject an agent that modulates the expression of MFSD12 or the activity of a gene product of MFSD12.

Some aspects of the disclosure are related to methods of treating or preventing cystinosis in a subject comprising administering to the subject an agent that modulates (e.g., inhibits) the expression of MFSD12 or the activity of a gene product of MFSD12.

In some embodiments, administration of the agent stabilizes (e.g., the rate of increase or decrease of a cysteine level is reduced or stopped or fluctuations in cysteine levels are reduced or eliminated) the level of cysteine in lysosomes of the subject. In some embodiments, administration of the agent increases the level of cysteine in lysosomes of the subject. In some embodiments, administration of the agent decreases the level of cysteine in lysosomes of the subject. In some embodiments, administration of the agent returns the level of cysteine in lysosomes of the subject to wild-type levels. As used herein, “lysosomes” are membrane-bound, acidic, cytoplasmic organelles responsible for intracellular protein degradation (Huizing et al., 2008).

Some aspects of the disclosure are related to methods of treating or preventing a disease, disorder, or dysfunction associated with an aberrant level of cysteine in a melanosome in a cell of a subject. The methods comprise, administering to the subject an agent that modulates the expression of MFSD12 or the activity of a gene product of MFSD12. In some embodiments, administration of the agent increases the level of cysteine in melanosomes of the subject. In some embodiments, administration of the agent decreases the level of cysteine in melanosomes of the subject. In some embodiments, administration of the agent stabilizes the level of cysteine in melanosomes of the subject. In some embodiments, administration of the agent returns the level of cysteine in melanosomes of the subject to wild-type levels. As used herein, “melanosomes” are specialized lysosome-related organelles (LROs) responsible for synthesis and storage of melanin in melanocytes.

In some embodiments, a subject is administered an effective amount of an agent to modulate the expression of MFSD12 or the activity of a gene product of MFSD12. The agent is not limited and may be any agent described herein. In some embodiments, the agent comprises a polypeptide, amino acid, oligonucleotide, lipid, carbohydrate, hybrid molecule, peptide, nucleic acid, or small molecule.

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered in a single dose, or through use of multiple doses, in various embodiments. A biological effect may be, e.g., reducing expression or activity of one or more gene products, reducing activity of a metabolic pathway or reaction, or reducing cell proliferation or survival of cells.

A single additional agent or multiple additional agents or treatment modalities may be co-administered (at the same or differing time points and/or via the same or differing routes of administration and/or on the same or a differing dosing schedule).

As used herein, a “subject” is a mammal, including but not limited to a primate (e.g., a human), rodent (e.g., mouse or rat) dog, cat, horse, cow, pig, sheep, goat, or chicken. Preferred subjects are human subjects. The human subject may be a pediatric or adult subject. In some embodiments, the subject is elderly. In some embodiments, the subject is at least about 40 years old, at least about 45 years old, at least about 50 years old, at least about 55 years old, at least about 60 years old, at least about 65 years old, at least about 70 years old, at least about 75 years old, at least about 80 years old, at least about 85 years old, or at least about 90 years old. In some embodiments, the subject has been diagnosed with, is suspected of having, or is at risk of having a disease or disorder described herein. In some embodiments, the subject has a lysosomal storage disease or disorder. In some embodiments, the subject has cystinosis. In some embodiments, the subject has a melanosomal disease or disorder.

In some embodiments, the disease or disorder is a lysosomal storage disease or disorder. The type of lysosomal storage disease is not limited. Lysosomal storage diseases, as used herein, shall mean any disorder characterized by reduced or absent lysosomal enzyme activity. Non-limiting examples of lysosomal storage diseases include Cystinosis, Pompe disease, including adult-onset/late-onset glycogen storage disease II, Gaucher disease, Fabry disease, Schindler disease, Niemann-Pick disease (including Type A, Type B, and Type C), Morquio disease (including Type A and Type B), Batten disease, Maroteaux-Lamy disease, metachromatic leukodystrophy disease, Hunter Syndrome, and Hurler Syndrome (including Hurler-Scheie Syndrome). Additional representative lysosomal storage diseases that may be treated using the disclosed methods include Activator Deficiency/GM2 Gangliosidosis, Alpha-mannosidosis, Aspartylglucosaminuria, Cholesteryl ester storage disease, Chronic Hexosaminidase A Deficiency, Danon disease, Farber disease, Fabry disease, Fucosidosis, Galactosialidosis (including Type I, Type II, and Type III), GM1 gangliosidosis, I-Cell disease/Mucolipidosis II, Infantile Free Sialic Acid Storage Disease/IS SD, Juvenile Hexosaminidase A Deficiency, Krabbe disease, Lysosomal acid lipase deficiency, Mucopolysaccharidoses disorders, Pseudo-Hurler polydystrophy/Mucolipidosis IIIA, Scheie Syndrome, Sanfilippo syndrome (including Type A, Type B, Type C, and Type D), MPS VII Sly Syndrome, mucopolysaccharidosis type I, mucopolysaccharidosis type II, Mucolipidosis I/Sialidosis, Mucolipidosis IIIC, Mucolipidosis type IV, Multiple sulfatase deficiency, Neuronal Ceroid Lipofuscinoses, CLN6 disease, Finnish Variant Late Infantile CLN5, Jansky-Bielschowsky disease/Late infantile CLN2/TPP1 Disease, Kufs/Adult-onset NCL/CLN4 disease, Northern Epilepsy/variant late infantile CLN8, Santavuori-Haltia/Infantile CLN1/PPT disease, Beta-mannosidosis, Pycnodysostosis, Sandhoff disease/Adult Onset/GM2 Gangliosidosis, Sandhoff disease/GM2 gangliosidosis, Salla disease/Sialic Acid Storage Disease, Tay-Sachs/GM2 gangliosidosis, and Wolman disease. In some embodiments, the lysosomal storage disease is cystinosis.

In some embodiments, methods of treating cystinosis include administering to the subject an agent that inhibits the expression of MFSD12 or the activity of a gene product of MFSD12. In some embodiments, the administration of the agent decreases the level of cysteine in lysosomes of the subject.

“Cystinosis” is a lysosomal storage disease characterized by abnormal accumulation of cystine (e.g., cysteine in the form of cystine). The disease results in deposition of crystals throughout the body, and, if untreated, it leads to failure to thrive, profound metabolic imbalance, early end-stage renal disease, thyroid failure, and multiorgan dysfunction. See Gahl et al., “Cystinosis.” N Engl J Med 2002; 347:111-121. Treatment primarily consists of administration of cysteamine. The CTNS gene (which codes for cystinosin) is the lysosomal cystine exporter mutated in cystinosis. In healthy individuals, mixed leukocyte preparations contain less than 0.2 nmol of half-cystine per milligram of protein, whereas in patients with nephropathic cystinosis, the values exceed 2.0 nmol of half-cystine per milligram of protein. Cystine content is expressed in units of half-cystine because initial methods of quantification involved a reduction of cystine followed by an assay for cysteine. The presence of typical corneal crystals on slit-lamp examination is also diagnostic of cystinosis, although crystals may be absent before one year of age. The target leukocyte cystine content is less than 1.0 nmol of half-cystine per milligram of protein. If this is not achieved, a dose of cysteamine is gradually increased until cystine depletion is satisfactory or side effects limit further increases.

It has been found that the physiological importance of a “cysteine specific lysosomal transport system” may be to aid lysosomal proteolysis by delivering cysteine into the lysosomal compartment. This maintains the catalytic activity of thiol-dependent lysosomal enzymes and breaks protein disulfide bridges at susceptible linkages, thereby allowing proteins to unfold, facilitating their degradation (Pisoni et al., 1990).

In some embodiments, the disease or disorder is a melanosomal disease or disorder. The type of melanosomal disease or disorder is not limited. Melanosomal diseases, as used herein, shall mean any disorder characterized by reduced or absent melanosomal enzyme activity. Non-limiting examples of disorders of melanosome biogenesis include Hermansky Pudlak Syndrome (HPS) and Chediak-Higashi Syndrome (CHS). A non-limiting example of a disorder of melanosome transport and/or transfer includes Griscelli Syndrome (GS) (Sitaram et al, 2012).

Some aspects of the disclosure are related to a method of altering skin pigmentation of a subject. The methods comprise topically administering to the subject an agent that modulates the expression of MFSD12 or the activity of a gene product of MFSD12 in the subject. In some embodiments, administration of the agent inhibits pheomelanin synthesis. In some embodiments, administration of the agent decreases the level of cysteine in melanosomes of the subject. In some embodiments, the agent comprises a polypeptide, amino acid, oligonucleotide, lipid, carbohydrate, hybrid molecule, peptide, nucleic acid, or small molecule. In some embodiments, the expression of MFSD12 or the activity of a gene product of MFSD12 is decreased, thereby darkening skin pigmentation of the subject.

MFSD12 may code for a lysosomal protein that modifies pigmentation in human melanocytes, with decreased MFSD12 expression being associated with darker pigmentation (Crawford et al., 2017). Variation in skin pigmentation may be due to the type and quantity of melanins generated, melanosome size, and the manner in which keratinocytes sequester and degrade melanins (Crawford et al., 2017). As used herein, “melanogenesis” means the production of melanin pigments; these are most often produced by cells called melanocytes (D.Mello et al., 2016). As used herein, “melanocyte” means a mature melanin-forming cell, especially in skin. Melanin in skin has two forms: eumelanin (predominant pigments found in dark skin and black hair) and pheomelanin (associated with red hair/freckled skin photype) (Yamaguchi et al, 2007).

Dermatological disorders (e.g., skin pigmentation disorders) may indicate an increased amount of melanin, leading to a darker color of the skin, called hypermelanosis or hyperpigmentation. Decreased or absent pigment results in the skin appearing lighter or white, known as hypomelanosis or hypopigmentation. The disorders can be genetic or acquired. In addition, there may be mixed hyper/hypopigmentation.

The type of skin pigmentation disorder is not limited. Non-limiting examples of hyperpigmentation disorders include: melasma (chloasma), Albright's syndrome, neurofibromatosis, freckles, and Addison's disease. Non-limiting examples of hypopigmentation disorders include: albinism (including ocular albinism (OA) as well as oculocutaneous albinisms that involve hypomorphic mutations (e.g. OCA1B)), vitiligo, piebaldism, and tuberous sclerosis. A non-limiting example of a mixed hyper/hypopigmentation disorder includes dyschromatosis symmetrica hereditaria (Yamaguchi et al., 2014).

As used herein, “treatment” or “treating”, in reference to a subject, includes amelioration, cure, and/or maintenance of a cure (i.e., the prevention or delay of relapse and/or reducing the likelihood of recurrence) of a disorder. Treatment after a disorder has started aims to reduce, ameliorate or altogether eliminate the disorder, and/or its associated symptoms, to prevent it from becoming worse, to slow the rate of progression, or to prevent the disorder from re-occurring once it has been initially eliminated (i.e., to prevent a relapse). Treating encompasses administration of an agent that may not have an effect on the disorder by itself but increases the efficacy of a second agent administered to the subject. A suitable dose and therapeutic regimen may vary depending upon the specific agent used, the mode of delivery of the compound, and whether it is used alone or in combination.

The dosage, administration schedule and method of administering the agent are not limited. In certain embodiments a reduced dose may be used when two or more agents are administered in combination either concomitantly or sequentially. The absolute amount will depend upon a variety of factors including other treatment(s), the number of doses and the individual patient parameters including age, physical condition, size and weight. These are factors well known to those of ordinary skill in the art and can be addressed with no more than routine experimentation. In some embodiments, a maximum tolerated dose may be used, that is, the highest safe and tolerable dose according to sound medical judgment. In some embodiments, a subject having a lysosomal storage disease or disorder (e.g., cystinosis) is administered cysteamine in combination with the agent. Cysteamine is an amino thiol drug mainly used in the treatment of cystinosis. Cysteamine is a cystine-depleting agent and it can prevent the buildup of cystine crystals in the kidneys in its oral form.

As used herein, pharmaceutical compositions comprise one or more agents or compositions that have therapeutic utility, and a pharmaceutically acceptable carrier, e.g., a carrier that facilitates delivery of agents or compositions. Agents and pharmaceutical compositions disclosed herein may be administered by any suitable means such as topically, orally, intranasally, subcutaneously, intramuscularly, intravenously, intra-arterially, parenterally, intraperitoneally, intrathecally, intratracheally, ocularly, sublingually, vaginally, rectally, dermally, or as an aerosol.

Also disclosed herein are pharmaceutical compositions which may be administered topically using one or more pharmaceutically-acceptable carriers or vehicles. Preferably, the pharmaceutical compositions disclosed herein can be applied directly to the skin of a subject. Also disclosed herein are topically administered compositions such as creams, ointments, serums, oils, lotions, gels, suspensions and/or solutions or the like that maintain a desired stability (e.g., remain stable at room temperature for at least two years or following exposure to freeze/thaw cycling).

Depending upon the type of condition to be treated, compounds of the invention may, for example, be inhaled, ingested or administered by systemic routes. Thus, a variety of administration modes, or routes, are available. The particular mode selected will typically depend on factors such as the particular compound selected, the particular condition being treated and the dosage required for therapeutic efficacy. The methods described herein, generally speaking, may be practiced using any mode of administration that is medically acceptable, meaning any mode that produces acceptable levels of efficacy without causing clinically unacceptable adverse effects. Preferred modes of administration are parenteral and oral routes. The term “parenteral” includes subcutaneous, intravenous, intramuscular, intraperitoneal, and intrasternal injection, or infusion techniques. In some embodiments, inhaled medications are of particular use because of the direct delivery to the lung, for example in lung cancer patients. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. In some embodiments agents are delivered by pulmonary aerosol. Other appropriate routes will be apparent to one of ordinary skill in the art.

Compositions

Some aspects of the disclosure are related to compositions comprising an agent that modulates the expression of MFSD12 or the activity of a gene product of MFSD12 when administered to a subject. The agent may be any agent, or combination of agents, disclosed herein.

In some embodiments, the agent increases the expression of MFSD12 or the activity of a gene product of MFSD12. In some embodiments, the agent decreases the expression of MFSD12 or the activity of a gene product of MFSD12. In some embodiments, the agent stabilizes the level of cysteine (e.g., the rate of increase or decrease of a cysteine level in a lysosome or melanosome is reduced or stopped, fluctuations in cysteine levels are reduced or eliminated). In some embodiments, the composition further comprises a second agent. In some embodiments, the composition further comprises cysteamine. In some embodiments, the composition is a cosmetic composition. In some embodiments, the composition is a topical cosmetic composition. In certain embodiments, the composition is useful for the treatment of dermatological disorders, such as skin pigmentation disorders.

In addition to the active agent(s), the pharmaceutical compositions typically comprise a pharmaceutically-acceptable carrier. The term “pharmaceutically-acceptable carrier”, as used herein, means one or more compatible solid or liquid vehicles, fillers, diluents, or encapsulating substances which are suitable for administration to a human or non-human animal. In preferred embodiments, a pharmaceutically-acceptable carrier is a non-toxic material that does not interfere with the effectiveness of the biological activity of the active ingredients. The term “compatible”, as used herein, means that the components of the pharmaceutical compositions are capable of being comingled with an agent, and with each other, in a manner such that there is no interaction which would substantially reduce the pharmaceutical efficacy of the pharmaceutical composition under ordinary use situations. Pharmaceutically-acceptable carriers should be of sufficiently high purity and sufficiently low toxicity to render them suitable for administration to the human or non-human animal being treated.

Some examples of substances which can serve as pharmaceutically-acceptable carriers are pyrogen-free water; isotonic saline; phosphate buffer solutions; sugars such as lactose, glucose, and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobrama; polyols such as propylene glycol, glycerin, sorbitol, mannitol, and polyethylene glycol; sugar; alginic acid; cocoa butter (suppository base); emulsifiers, such as the Tweens; as well as other non-toxic compatible substances used in pharmaceutical formulation. Wetting agents and lubricants such as sodium lauryl sulfate, as well as coloring agents, flavoring agents, excipients, tableting agents, stabilizers, antioxidants, and preservatives, can also be present. It will be appreciated that a pharmaceutical composition can contain multiple different pharmaceutically acceptable carriers.

A pharmaceutically-acceptable carrier employed in conjunction with the compounds described herein is used at a concentration or amount sufficient to provide a practical size to dosage relationship. The pharmaceutically-acceptable carriers, in total, may, for example, comprise from about 60% to about 99.99999% by weight of the pharmaceutical compositions, e.g., from about 80% to about 99.99%, e.g., from about 90% to about 99.95%, from about 95% to about 99.9%, or from about 98% to about 99%.

Pharmaceutically-acceptable carriers suitable for the preparation of unit dosage forms for oral administration and topical application are well-known in the art. Their selection will depend on secondary considerations like taste, cost, and/or shelf stability, which are not critical for the purposes of the subject invention, and can be made without difficulty by a person skilled in the art.

Pharmaceutically acceptable compositions (including cosmetic preparations) can include diluents, fillers, salts, buffers, stabilizers, solubilizers and other materials which are well-known in the art. The choice of pharmaceutically-acceptable carrier to be used in conjunction with the compounds of the present invention is basically determined by the way the compound is to be administered. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof in certain embodiments. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. It will also be understood that a compound can be provided as a pharmaceutically acceptable pro-drug, or an active metabolite can be used. Furthermore it will be appreciated that agents may be modified, e.g., with targeting moieties, moieties that increase their uptake, biological half-life (e.g., pegylation), etc.

The agents may be administered in pharmaceutically acceptable solutions, which may routinely contain pharmaceutically acceptable concentrations of salt, buffering agents, preservatives, compatible carriers, adjuvants, and optionally other therapeutic ingredients.

The agents may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

For oral administration, compositions can be formulated readily by combining the active agent(s) with pharmaceutically acceptable carriers well known in the art. Such carriers enable the agents to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In certain embodiments, the vehicle is a biocompatible microparticle or implant that is suitable for implantation into the mammalian recipient. Exemplary bioerodible implants that are useful in accordance with this method are described in PCT International Application Publication No. WO 95/24929, entitled “Polymeric Gene Delivery System”, which reports on a biodegradable polymeric matrix for containing a biological macromolecule. The polymeric matrix may be used to achieve sustained release of the agent in a subject. In some embodiments, an agent described herein may be encapsulated or dispersed within a biocompatible, preferably biodegradable polymeric matrix. The polymeric matrix may be in the form of a microparticle such as a microsphere (wherein the agent is dispersed throughout a solid polymeric matrix) or a microcapsule (wherein the agent is stored in the core of a polymeric shell). Other forms of polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the agents may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems can include time-release, delayed release or sustained release delivery systems. Such systems can avoid repeated administrations of the peptide, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the platelet reducing agent is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation. Liposomes, for example, which may comprise phospholipids or other lipids, are nontoxic, physiologically acceptable carriers that may be used in some embodiments. Liposomes can be prepared according to methods known to those skilled in the art. In some embodiments, for example, liposomes may be prepared as described in U.S. Pat. No. 4,522,811. Liposomes, including targeted liposomes, pegylated liposomes, and polymerized liposomes, are known in the art (see, e.g., Hansen C B, et al., Biochim Biophys Acta. 1239(2):133-44, 1995; Torchilin V P, et al., Biochim Biophys Acta, 1511(2):397-411, 2001; Ishida T, et al., FEBS Lett. 460(1):129-33, 1999). In some embodiments, a lipid-containing particle may be prepared as described in any of the following PCT application publications, or references therein: WO/2011/127255; WO/2010/080724; WO/2010/021865; WO/2010/014895; WO2010147655.

Long-term release, as used herein, means that the implant is constructed and arranged to deliver therapeutic levels of the active agent for at least 30 days, and preferably 60 days. Long-term sustained release implants are well-known to those of ordinary skill in the art and include some of the release systems described above.

In some embodiments, it may be advantageous to formulate oral or parenteral compositions in dosage unit form for ease of administration and uniformity of dosage. Unit dosage form as used herein refers to physically discrete units suited as unitary dosages for the subject to be treated; each unit containing a predetermined quantity of active compound calculated to produce the desired therapeutic effect in association with the required pharmaceutical carrier.

Generally, treatment of a subject can include a single treatment or, in many cases, can include a series of treatments. A pharmaceutical composition can be administered at various intervals and over different periods of time as required, e.g., multiple times per day, daily, every other day, once or more a week for between about 1 to 10 weeks, between 2 to 8 weeks, between about 3 to 7 weeks, about 4, 5, or 6 weeks, etc. It will be appreciated that multiple cycles of administration may be performed. Numerous variations are possible. The skilled artisan will appreciate that certain factors can influence the dosage and timing required to effectively treat a subject, including but not limited to the severity of the disease or disorder, previous treatments, the general health and/or age of the subject, and other diseases present.

Screening Methods

Some aspects of the disclosure are related to methods of identifying a candidate agent that modulates the expression of MFSD12 or the activity of a gene product of MFSD12 in a lysosome and/or in a melanosome.

In some embodiments, the method comprises contacting the lysosome and/or melanosome with a test agent. The levels of cysteine in the lysosome and/or melanosome may then be measured, and the agent may be identified as an inhibitor or activator of expression of MFSD12 or the activity of a gene product of MFSD12. In some embodiments, the test agent is identified as an inhibitor of expression of MFSD12 or the activity of a gene product of MFSD12 if the level of cysteine in the lysosome and/or melanosome is lower than a reference level. In some embodiments, the test agent is identified as an agent that increases expression of MFSD12 or the activity of a gene product of MFSD12 if the level of cysteine in the lysosome and/or in the melanosome is higher than a reference level. In some embodiments, the reference level is the level of cysteine in lysosomes and/or in melanosomes under equivalent conditions but not exposed to the test agent. In some embodiments, the agent comprises a polypeptide, amino acid, oligonucleotide, lipid, carbohydrate, hybrid molecule, peptide, nucleic acid, or small molecule. In some embodiments, the test agent is capable of increasing the level of expression of MFSD12 or the activity of a gene product of MFSD12 by at least 10%, 25%, 75%, or 100%. In some embodiments, the test agent is capable of increasing the level of expression of MFSD12 or the activity of gene product of MFSD12 by at least 2 fold, at least 3 fold, at least 5 fold, at least 10 fold, at least 25 fold, at least 50 fold, at least 75 fold, or at least 100 fold. In some embodiments, the test agent is capable of decreasing the level of expression of MFSD12 or the activity of a gene product of MFSD12 by at least 10%, 25%, 50%, 75%, 80%, 85%, 90%, or 95%.

Methods of detecting cysteine are known in the art and are not limited. In some embodiments, the method is performed in isolated lysosomes. In some embodiments, the lysosomes are present in one or more cells. In some embodiments, the lysosomes are isolated from one or more cells after contact with the test agent. In some embodiments, the method is performed in isolated melanosomes. In some embodiments, the melanosomes are present in one or more cells. In some embodiments, the melanosomes are isolated from one or more cells after contact with the test agent.

In some embodiments, an inhibitor of MFSD12 expression (e.g., an expression product of the MFSD12 gene) is identified by contacting a cell with a test agent and measuring the level of MFSD12 mRNA or protein. The agent is identified as an inhibitor of MFSD12 expression if the agent reduces the level of MFSD12 mRNA or protein as compared to a reference level (e.g., a control cell).

In some embodiments, the methods of screening test agents described herein further comprise contacting the identified MFSD12 modulator with a test cell and measuring proliferation and/or survival of the contacted test cell as compared to a control cell not contacted with the identified modulator.

In some embodiments a method of screening one or more test agents to identify a modulator of MFSD12 expression or activity comprises a high-throughput transport assay (e.g., in vitro transport assay). In some aspects an artificial membrane (e.g., a liposome) may be utilized. In other aspects a bacterial system may be utilized (e.g., Gram-negative bacteria such as E. coli or Gram-positive bacteria such as B. subtilis or Lactococcus lactis). In some embodiments, MFSD12 may be reconstituted into a liposome and one or more test agents are applied to MFSD12. In some embodiments bacterial Lactococcus lactis cells are grown to express MFSD12 and the cells are contacted with one or more test agents. In some aspects the cysteine transport activity of MFSD12 (e.g., transport of the cysteine into the liposome or the cell by the MFSD12) is measured.

In certain embodiments of any method described herein, the survival or proliferation of cells, e.g., test cells and/or control cells, is determined by an assay selected from: a cell counting assay, a replication labeling assay, a cell membrane integrity assay, a cellular ATP-based viability assay, a mitochondrial reductase activity assay, a caspase activity assay, an Annexin V staining assay, a DNA content assay, a DNA degradation assay, and a nuclear fragmentation assay. Exemplary assays include BrdU, EdU, or H3-Thymidine incorporation assays; DNA content assays using a nucleic acid dye, such as Hoechst Dye, DAPI, actinomycin D, 7-aminoactinomycin D or propidium iodide; cellular metabolism assays such as AlamarBlue, MTT, XTT, and CellTitre Glo; nuclear fragmentation assays; cytoplasmic histone associated DNA fragmentation assay; PARP cleavage assay; TUNEL staining; and Annexin staining. In some embodiments, gene expression analysis (e.g., microarray, cDNA array, quantitative RT-PCR, RNAse protection assay, RNA-Seq) may be used to measure the expression of genes whose products mediate or are correlated with cell cycle, cell survival (or cell death, e.g., apoptosis), and/or cell proliferation, as an indication of the effect of an agent on cell viability or proliferation. Alternately or additionally, expression of proteins encoded by such genes may be measured. In other embodiments, the activity of a gene, such as those disclosed herein, can be assayed in a compound screen. In some embodiments, cells are modified to comprise an expression vector that includes a regulatory region of a gene whose products mediate or are correlated with cell cycle, cell survival (or cell death), and/or cell proliferation operably linked to a sequence that encodes a reporter gene product (e.g., a luciferase enzyme), wherein expression of the reporter gene is correlated with transcriptional activity of the gene. In such embodiments assessment of reporter gene expression (e.g., luciferase activity) provides an indirect method for assessing cell survival or proliferation. Those of ordinary skill in the art are aware of genes whose products mediate or are correlated with cell cycle, cell survival (or cell death), and/or cell proliferation.

In some embodiments the activity of an agent (e.g., a test agent) can be tested by contacting test cells and control cells that are in a co-culture. Co-cultures enable selective evaluation of the properties (e.g., survival or proliferation) of two or more populations of cells (e.g., test and control cells) in contact with an agent in a common growth chamber. Typically, each population of cells grown in co-culture will have an identifying characteristic that is detectable and distinct from an identifying characteristic of the other population(s) of cells in the co-culture. In some embodiments, the identifying characteristic comprises a level of expression of a fluorescent protein or other reporter protein or a protein expressed at the cell surface that could be detected using an antibody. Numerous fluorescent proteins are known in the art and may be used. Such proteins include, e.g., green, blue, yellow, red, orange, and cyan fluorescent proteins (FP). In some embodiments, test cells and control cells express different, distinguishable FPs, e.g., a red FP and a green FP, or other pairs of FPs that have different emission spectra. Other reporter proteins include, e.g., enzymes such as luciferase, beta-galactosidase, alkaline phosphatase, etc. However, other identifying characteristics known in the art may be suitable, provided that the identifying characteristic enables measurement (e.g., by FACS or other suitable assay method) of the level of survival or proliferation of each of the two or more populations of cells in the co-culture. A cell can be modified to have an identifying characteristic using methods known in the art, e.g., by introducing into the cell a nucleic acid construct encoding an FP (or other detectable protein) operably linked to a promoter. In some embodiments, a nucleic acid construct that encodes an RNAi agent that reduces expression of MFSD12 and a nucleic acid construct that encodes a FP or other detectable protein are incorporated into the same vector. In some embodiments, they may be in different vectors. In some embodiments, the construct(s) may be integrated into the genome of the cell.

Compositions, e.g., co-cultures, comprising at least some test cells (e.g., between 1% and 99% test cells) and at least some control cells (e.g., between 1% and 99% control cells), are disclosed herein. In some embodiments the percentage of test cells is between 10% and 90%. In other embodiments the percentage of test cells is between 20% and 80%. In some embodiments the percentage of test cells is between 30% and 70%. In some embodiments the percentage of test cells is between 40% and 60%, e.g., about 50%. In some embodiments the composition further comprises a test agent.

In some embodiments, test cells and control cells are maintained in separate vessels (e.g., separate wells of a microwell plate) under substantially identical conditions.

Assay systems comprising test cells, control cells, and one or more test compounds, e.g., 10, 100, 1000, 10,000, or more test agents, wherein the cells and test agents are arranged in one or more vessels in a manner suitable for assessing effect of the test compound(s) on the cells, are aspects of the invention. Typically the vessels contain a suitable tissue culture medium, and the test compounds are present in the tissue culture medium. One of skill in the art can select a medium and culture environment appropriate for culturing a particular cell type.

In various embodiments the number of test agents is at least 10; 100; 1000; 10,000; 100,000; 250,000; 500,000 or more. In some embodiments test agents are tested in individual vessels, e.g., individual wells of a multiwell plate (sometimes referred to as microwell or microtiter plate or dish). In some embodiments a multiwell plate of use in performing an assay or culturing or testing cells or agents has 6, 12, 24, 96, 384, or 1536 wells. Cells (test cells and/or control cells) can be contacted with one or more test agents for varying periods of time and/or at different concentrations. In certain embodiments cells are contacted with test agent(s) for between 1 hour and 20 days, e.g., for between 12 and 48 hours, between 48 hours and 5 days, e.g., about 3 days, between 2 and 5 days, between 5 days and 10 days, between 10 days and 20 days, or any intervening range or particular value. Cells can be contacted with a test agent during all or part of a culture period. Cells can be contacted transiently or continuously. Test agents can be added to culture media at the time of replenishing the media and/or between media changes. If desired, test agent can be removed prior to assessing growth and/or survival. In some embodiments a test agent is tested at 1, 2, 3, 5, 8, 10 or more concentrations. Concentrations of test agent may range, for example, between about 1 nM and about 100 μM. For example, concentrations 1 nM, 10 nM, 50 nM, 100 nM, 500 nM, 1 μM, 5 μM, 10 μM, 50 μM, 100 μM (or any subset of the foregoing) may be used.

In some embodiments of any aspect or embodiment in the present disclosure relating to cells, a population of cells, cell sample, or similar terms, the number of cells is between 10 and 10¹³ cells. In some embodiments the number of cells may be at least about 10³, 10⁴, 10⁵, 10⁶, 10⁷, 10⁸, 10⁹, 10¹⁰, 10¹¹, 10¹² cells, or more. In some embodiments, the number of cells is between 10⁵ and 10¹² cells, e.g., at least 10⁶, 10⁷, 10⁸, 10⁹, 10¹⁰, 10¹¹, up to about 10¹² or about 10¹³. In some embodiments a screen is performed using multiple populations of cells and/or is repeated multiple times. In some embodiments, the number of cells is between 10⁵ and 10¹² cells, e.g., at least 10⁶, 10⁷, 10⁸, 10⁹, 10¹⁰, 10¹¹, up to about 10¹². In some embodiments smaller numbers of cells are of use, e.g., between 1-10⁴ cells. In some embodiments a population of cells is contained in an individual vessel, e.g., a culture vessel such as a culture plate, flask, or well. In some embodiments a population of cells is contained in multiple vessels. In some embodiments two or more cell populations are pooled to form a larger population.

In some embodiments, each of one or more test cells is contacted with a different concentration of, and/or for a different duration with, a test agent than at least one other test cell; and/or each of the one or more control cells is contacted with a different concentration of, and/or for a different duration with, the test agent than at least one other control cell.

In some embodiments, a high throughput screen (HTS) is performed. A high throughput screen can utilize cell-free or cell-based assays. High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarrón R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hüser.

The term “hit” generally refers to an agent that achieves an effect of interest in a screen or assay, e.g., an agent that has at least a predetermined level of modulating effect on cell survival, cell proliferation, gene expression, protein activity, or other parameter of interest being measured in the screen or assay. Test agents that are identified as hits in a screen may be selected for further testing, development, or modification. In some embodiments a test agent is retested using the same assay or different assays. Additional amounts of the test agent may be synthesized or otherwise obtained, if desired. Physical testing or computational approaches can be used to determine or predict one or more physicochemical, pharmacokinetic and/or pharmacodynamic properties of compounds identified in a screen. For example, solubility, absorption, distribution, metabolism, and excretion (ADME) parameters can be experimentally determined or predicted. Such information can be used, e.g., to select hits for further testing, development, or modification. For example, small molecules having characteristics typical of “drug-like” molecules can be selected and/or small molecules having one or more unfavorable characteristics can be avoided or modified to reduce or eliminate such unfavorable characteristic(s).

Additional compounds, e.g., analogs, that have a desired activity can be identified or designed based on compounds identified in a screen. In some embodiments structures of hit compounds are examined to identify a pharmacophore, which can be used to design additional compounds. An additional compound may, for example, have one or more altered, e.g., improved, physicochemical, pharmacokinetic (e.g., absorption, distribution, metabolism and/or excretion) and/or pharmacodynamic properties as compared with an initial hit or may have approximately the same properties but a different structure. For example, a compound may have higher affinity for the molecular target of interest, lower affinity for a non-target molecule, greater solubility (e.g., increased aqueous solubility), increased stability, increased bioavailability, oral bioavailability, and/or reduced side effect(s), modified onset of therapeutic action and/or duration of effect. An improved property is generally a property that renders a compound more readily usable or more useful for one or more intended uses. Improvement can be accomplished through empirical modification of the hit structure (e.g., synthesizing compounds with related structures and testing them in cell-free or cell-based assays or in non-human animals) and/or using computational approaches. Such modification can make use of established principles of medicinal chemistry to predictably alter one or more properties. An analog that has one or more improved properties may be identified and used in a composition or method described herein. In some embodiments a molecular target of a hit compound is identified or known. In some embodiments, additional compounds that act on the same molecular target may be identified empirically (e.g., through screening a compound library) or designed.

Data or results from testing an agent or performing a screen may be stored or electronically transmitted. Such information may be stored on a tangible medium, which may be a computer-readable medium, paper, etc. In some embodiments a method of identifying or testing an agent comprises storing and/or electronically transmitting information indicating that a test agent has one or more propert(ies) of interest or indicating that a test agent is a “hit” in a particular screen, or indicating the particular result achieved using a test agent. A list of hits from a screen may be generated and stored or transmitted. Hits may be ranked or divided into two or more groups based on activity, structural similarity, or other characteristics.

Once a candidate agent is identified, additional agents, e.g., analogs, may be generated based on it. An additional agent, may, for example, have increased cell uptake, increased potency, increased stability, greater solubility, or any improved property. In some embodiments a labeled form of the agent is generated. The labeled agent may be used, e.g., to directly measure binding of an agent to a molecular target in a cell. In some embodiments, a molecular target of an agent identified as described herein may be identified. An agent may be used as an affinity reagent to isolate a molecular target. An assay to identify the molecular target, e.g., using methods such as mass spectrometry, may be performed. Once a molecular target is identified, one or more additional screens maybe performed to identify agents that act specifically on that target.

Any of a wide variety of agents may be used as a test agent in various embodiments. For example, a test agent may be a small molecule, polypeptide, peptide, amino acid, nucleic acid, oligonucleotide, lipid, carbohydrate, or hybrid molecule. In some embodiments a nucleic acid used as a test agent comprises a siRNA, shRNA, antisense oligonucleotide, aptamer, or random oligonucleotide. In some embodiments a test agent is cell permeable or provided in a form or with an appropriate carrier or vector to allow it to enter cells.

Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. A compound library may comprise natural products and/or compounds generated using non-directed or directed synthetic organic chemistry. In some embodiments a library is a small molecule library, peptide library, peptoid library, cDNA library, oligonucleotide library, or display library (e.g., a phage display library). In some embodiments a library comprises agents of two or more of the foregoing types. In some embodiments oligonucleotides in an oligonucleotide library comprise siRNAs, shRNAs, antisense oligonucleotides, aptamers, or random oligonucleotides.

A library may comprise, e.g., between 100 and 500,000 compounds, or more. In some embodiments a library comprises at least 10,000, at least 50,000, at least 100,000, or at least 250,000 compounds. In some embodiments compounds of a compound library are arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not found in nature) or naturally occurring. In some embodiments compounds that have been identified as “hits” or “leads” in a drug discovery program and/or analogs thereof. In some embodiments a library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities such as the U.S. National Institutes of Health (NIH). In some embodiments a test agent is not an agent that is found in a cell culture medium known or used in the art, e.g., for culturing vertebrate, e.g., mammalian cells, e.g., an agent provided for purposes of culturing the cells. In some embodiments, if the agent is one that is found in a cell culture medium known or used in the art, the agent may be used at a different, e.g., higher, concentration when used as a test agent in a method or composition described herein.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### Example 1: Melanosomal Metabolomics Reveals a Cysteine Importer for Melanosomes and Lysosomes

Pigment producing cells like melanocytes synthesize melanin in membrane-bound, lysosome-related organelles (LRO) called melanosomes. Within melanosomes, tyrosinase catalyzes the oxidation of tyrosine to dopaquinone, which is then further oxidized and polymerized into a melanin pigment (D'Alba and Shawkey, 2019; Prota, 1992). While in cell-free systems tyrosinase, tyrosine, and oxygen are sufficient to produce simple melanin polymers, in vivo additional melanosomal proteins influence the chemical composition and quantity of the melanin synthesized. Many of these proteins have been implicated in human pigment variation, but in many cases their biochemical functions remain unknown or unclear (Basrur et al., 2003; Sturm, 2009). The current understanding of melanin synthesis has been in large part driven by the study of cell-free melanin synthesis reactions and the chemical degradation of isolated melanins (D'Alba and Shawkey, 2019; Prota, 1992). It was reasoned that the direct measurement of melanosomal metabolites would enable a better understanding of the function of melanosomal proteins in cells. However, established methods for isolating pure melanosomes are lengthy and would allow for metabolites of interest to oxidize or diffuse out of the organelle (Watabe et al., 2005).

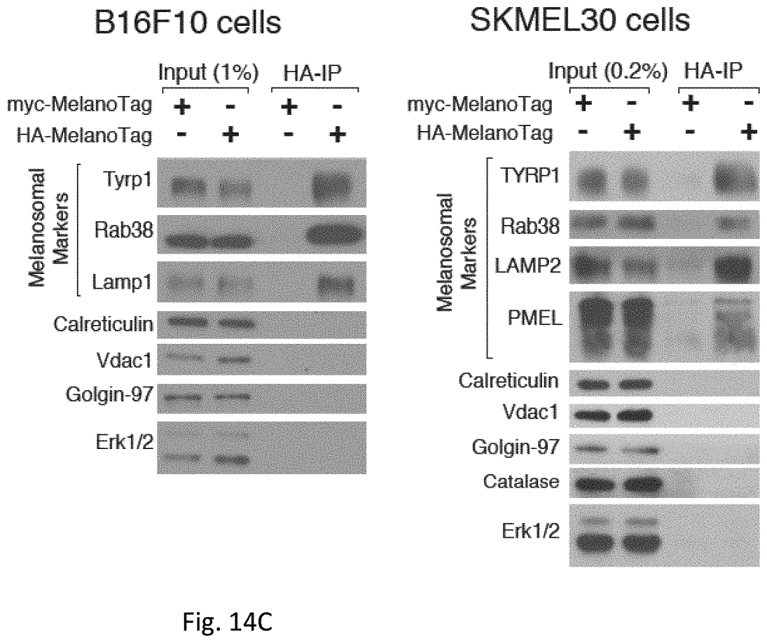

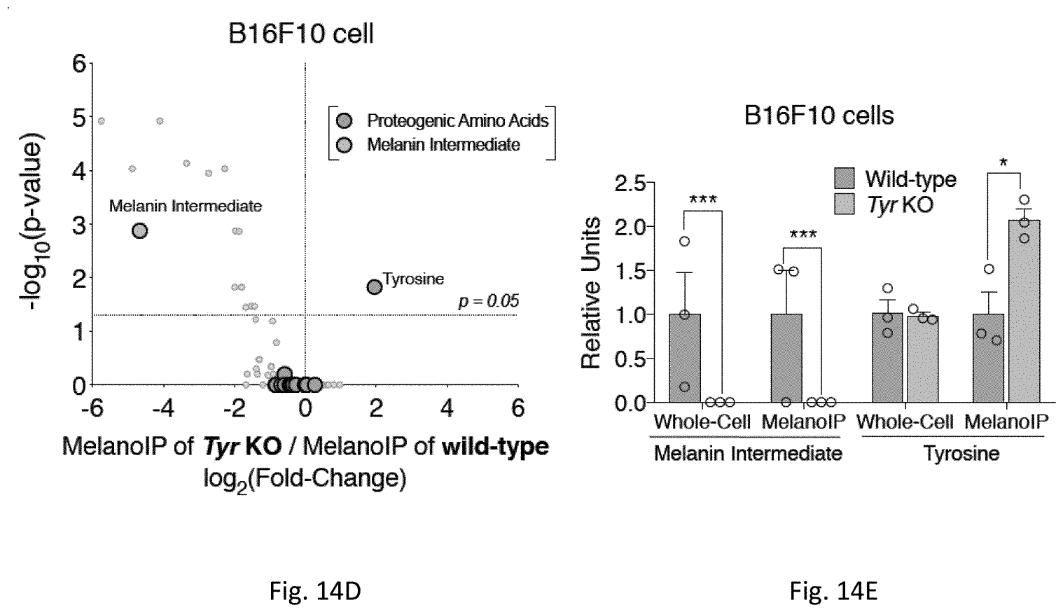

To overcome this challenge, a method for the rapid isolation of melanosomes was developed that is compatible with the metabolite profiling of their contents. This method, termed ‘MelanoIP,’ adapts previously developed techniques for the isolation of mitochondria and lysosomes in which recombinant proteins that serve as specific tags for organelles enable their rapid immunopurification (FIG. 14A) (Abu-Remaileh et al., 2017; Chen et al., 2016). A melanosome-specific tag (MelanoTag) was engineered based on the melanosomal marker GPR143 (Bruder et al., 2012) (FIG. 14A) and validated it co-localizes with the melanosomal protein Tyrp1 when expressed in cultured melanoma cells (FIG. 14B). The MelanolP method successfully purified melanosomes from murine B16F10 and human SKMEL30 melanoma cell lines as judged by the enrichment of melanosomal markers Tyrp1, PMEL, and Rab38 over of those for the Golgi, endoplasmic reticulum, and mitochondria (FIG. 14C). As many lysosomal proteins also localize to melanosomes (Basrur et al., 2003; Diment et al., 1995), it was not surprising that the purified melanosomes also contained the lysosomal marker proteins Lamp1 and LAMP2 (FIG. 14C). In SKMEL30 cells, the enrichment of processed PMEL could also be seen over its full length form, consistent with the method capturing the luminal contents of the organelles (Bissig et al., 2016).

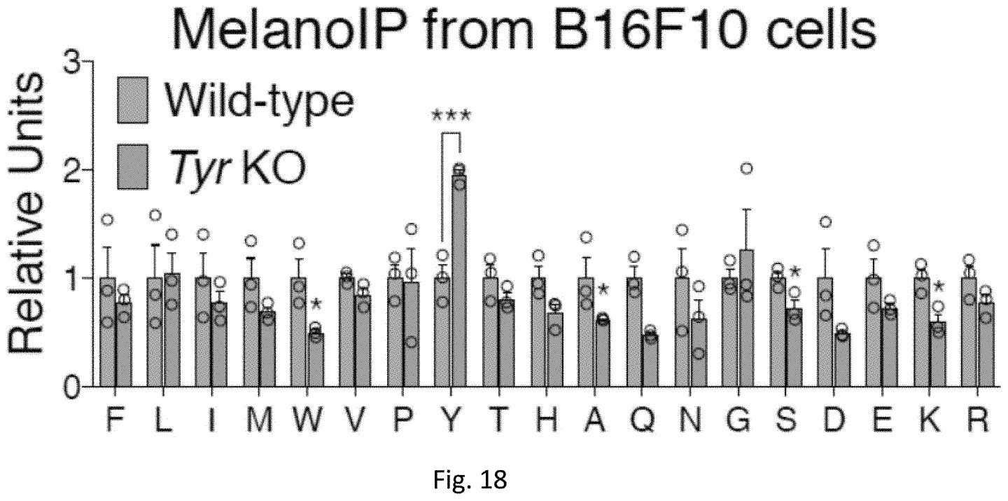

To validate the MelanolP approach, liquid chromatography mass-spectrometry (LC/MS) was used to profile metabolites in melanosomes from wild-type and Tyr-deficient B16F10 cells. Tyr encodes the aforementioned tyrosinase, an essential and rate-limiting enzyme in melanin synthesis. Unlike wild-type samples, Tyr knock-out cells and melanosomes lacked detectable melanin synthesis intermediates (FIGS. 14D-14E). Loss of tyrosinase did not impact whole-cell tyrosine levels, but did cause a 2-fold accumulation of tyrosine in melanosomes (FIGS. 14D-14E), consistent with tyrosine being the tyrosinase substrate. No other proteogenic amino acids were affected to the same degree (FIG. 14E and FIG. 18). These results highlight the value of the MelanolP for detecting compartmentalized changes in metabolites that have only a small fraction of their whole-cell pool represented in the melanosome.

The value of the MelanolP method was tested next for deorphaning a melanosomal protein of unknown function. MFSD12 was recently described as a gene in which loss of function mutations result in darker pigmentation (Adhikari et al., 2019; Crawford et al., 2017). This overall darkening is thought to occur because MFSD12 is required for the synthesis of red pheomelanin pigment, but not brown-black eumelanin pigment (Crawford et al., 2017). However, no direct biochemical function has been suggested for MFSD12, and it has been reported to localize mostly to lysosomes in cultured pigmented cells. Given that lysosomal proteins are often also melanosomal, it was hypothesized that a minor population of MFSD12 could exist at the melanosome and directly affect melanosomal metabolism.

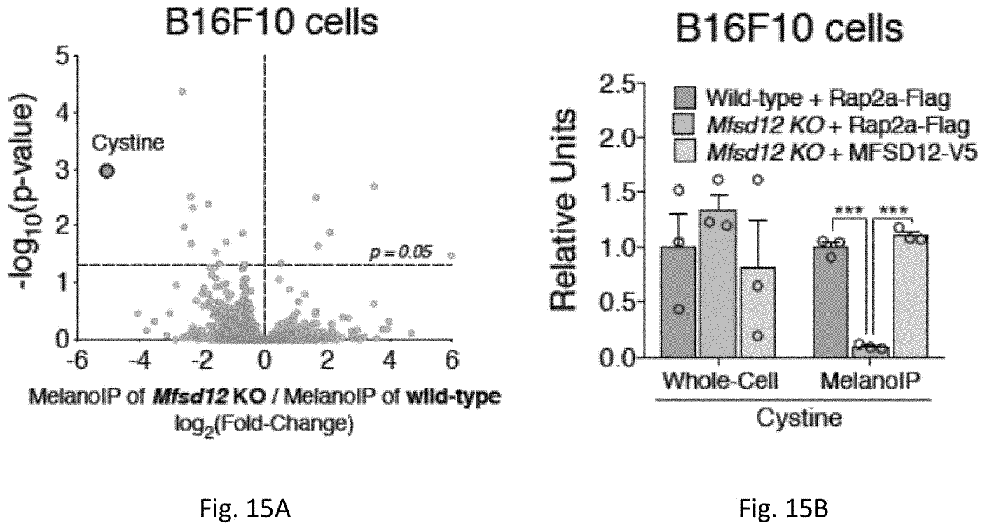

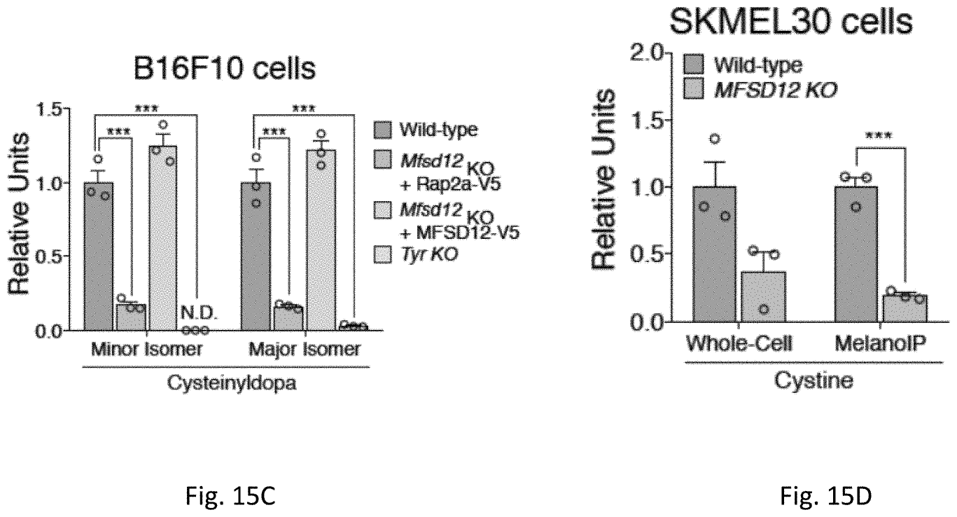

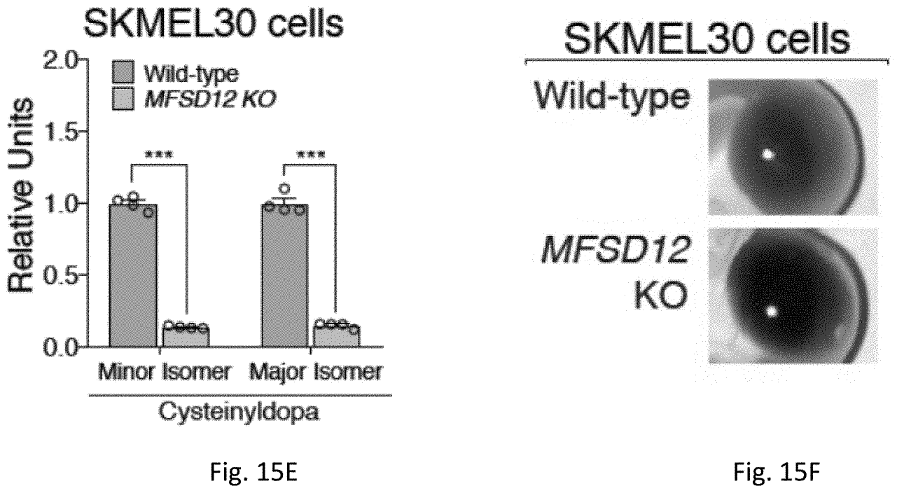

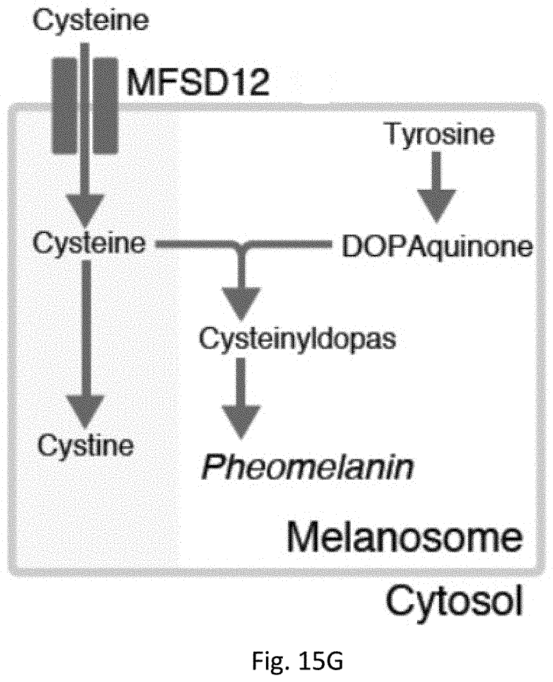

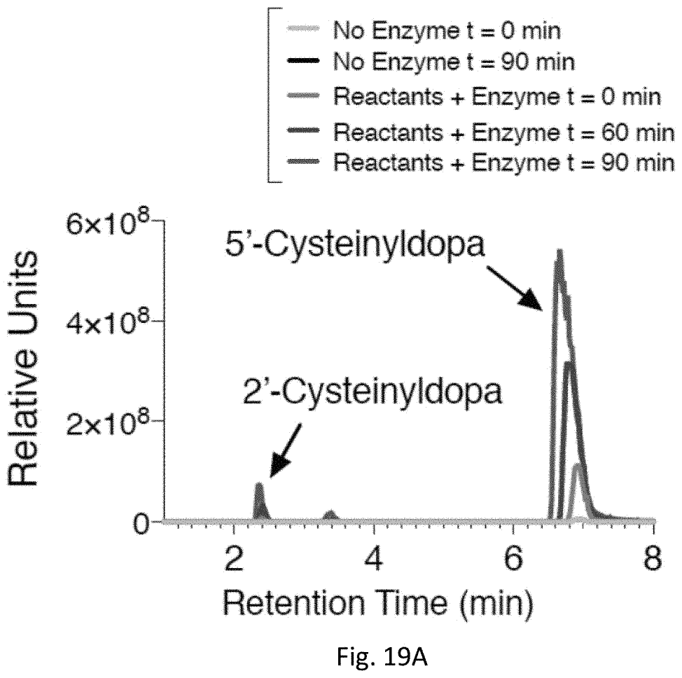

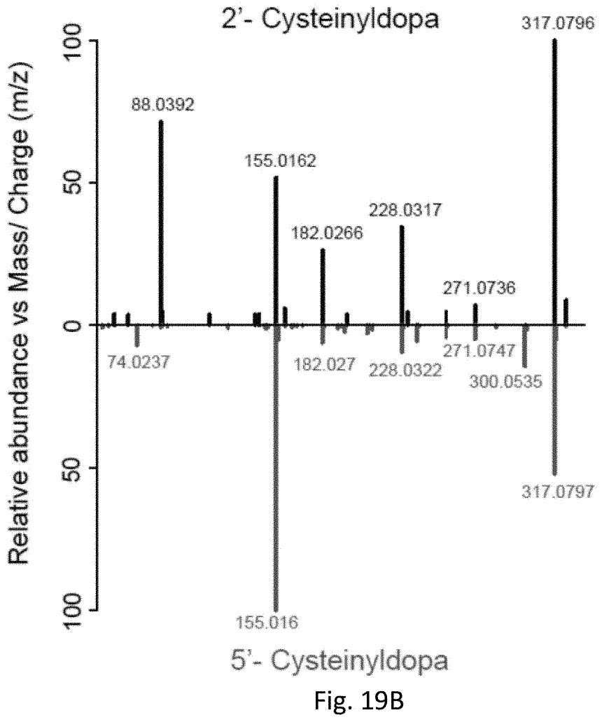

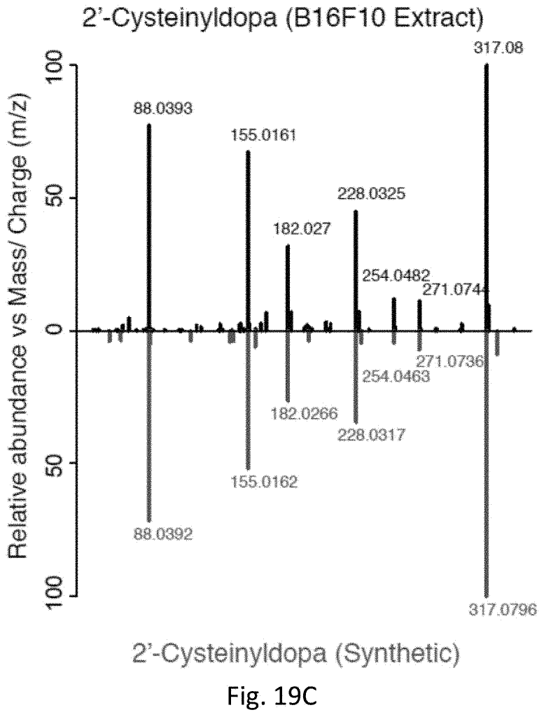

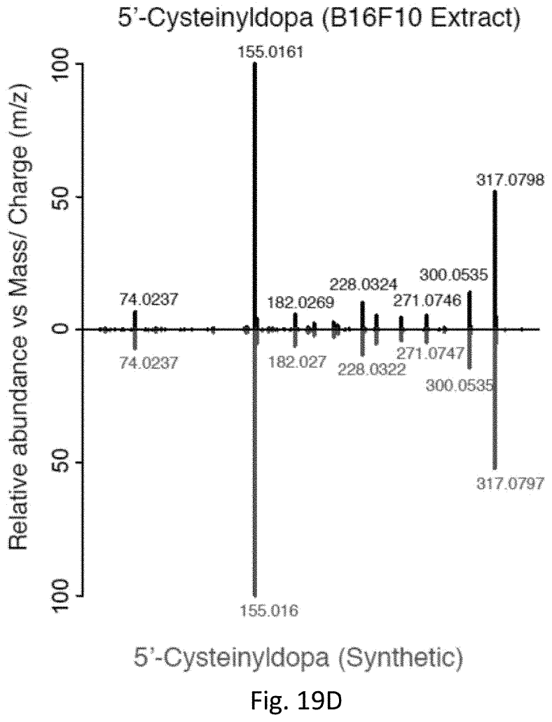

MFSD12 is part of the large MFS superfamily of 12-transmembrane domain transporters (Yan, 2015), suggesting it has an unknown transport activity. Untargeted metabolite profiling of melanosomes from wild-type and Mfsd12 knock-out B16F10 melanoma cells revealed that loss of MFSD12 greatly reduced melanosomal cystine levels (FIG. 15A). In follow-up studies, cystine dropped 11-fold in melanosomes from B16F10 cells upon loss of MFSD12, a phenotype fully reversed by its re-expression (FIG. 15B). It was reasoned that the decrease in cystine, the oxidized dimeric form of cysteine, might result from a decrease in cysteine in melanosomes. Consistent with this possibility, loss of MFSD12 also greatly reduced the levels of cysteinyldopas (FIG. 15C, FIGS. 19A-19D), which are precursors for pheomelanin synthesis and made in melanosomes by the co-oxidation of cysteine and tyrosine (D'Alba and Shawkey, 2019; Prota, 1992). Even with chemical derivatization cysteine in melanosomes could not be detected, likely because of its rapid conversion to cystine and the cysteinyldopas. Gratifyingly, like in the murine cells, in human SKMEL30 cells loss of MFSD12 also reduced melanosomal cystine and cysteinyldopas (FIGS. 15D-15E). Interestingly, the MFSD12-null SKMEL30 cells are visibly darker than their wild-type counterparts, as are the MFSD12 mutant mice (FIG. 15F) (Bloom and Falconer, 1966; Crawford et al., 2017). It has been known that isolated melanosomes can influx cysteine, but the responsible transporter has not yet been identified (Potterf et al., 1999). The results indicated MFSD12 is a component of this melanosomal cysteine import system (FIG. 15G).

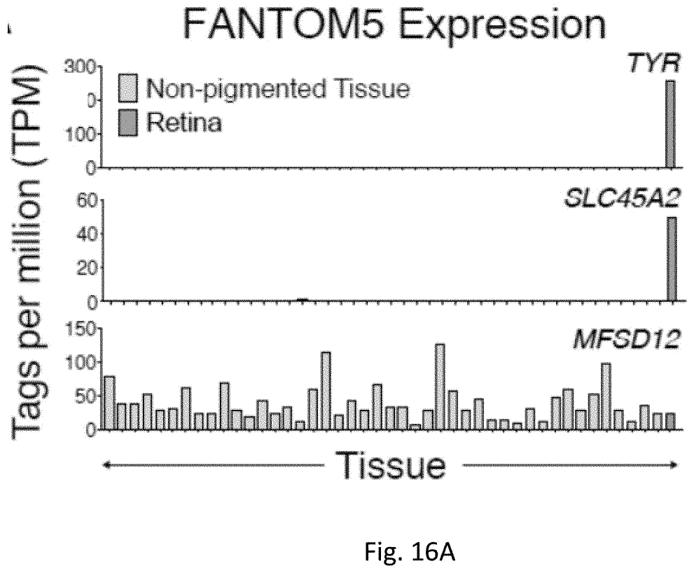

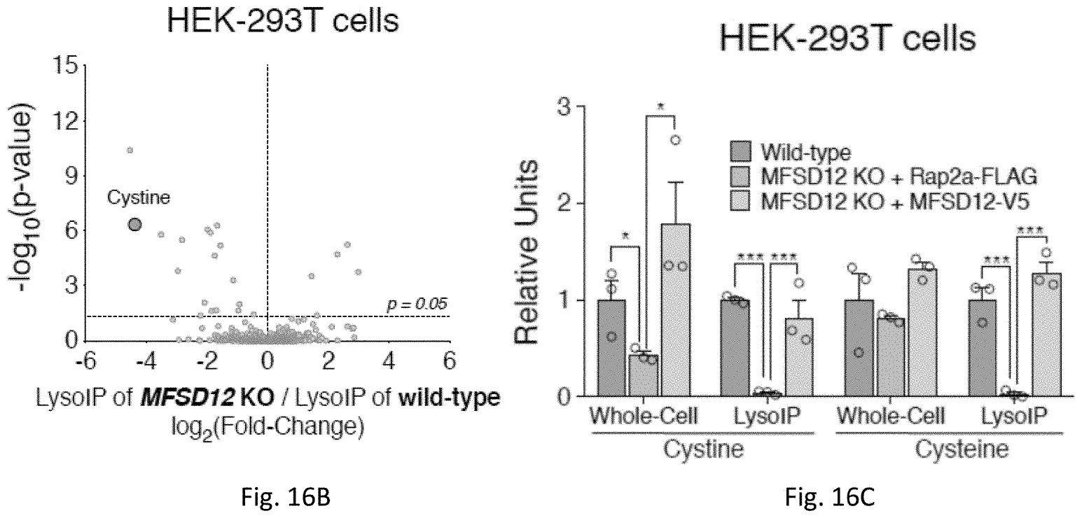

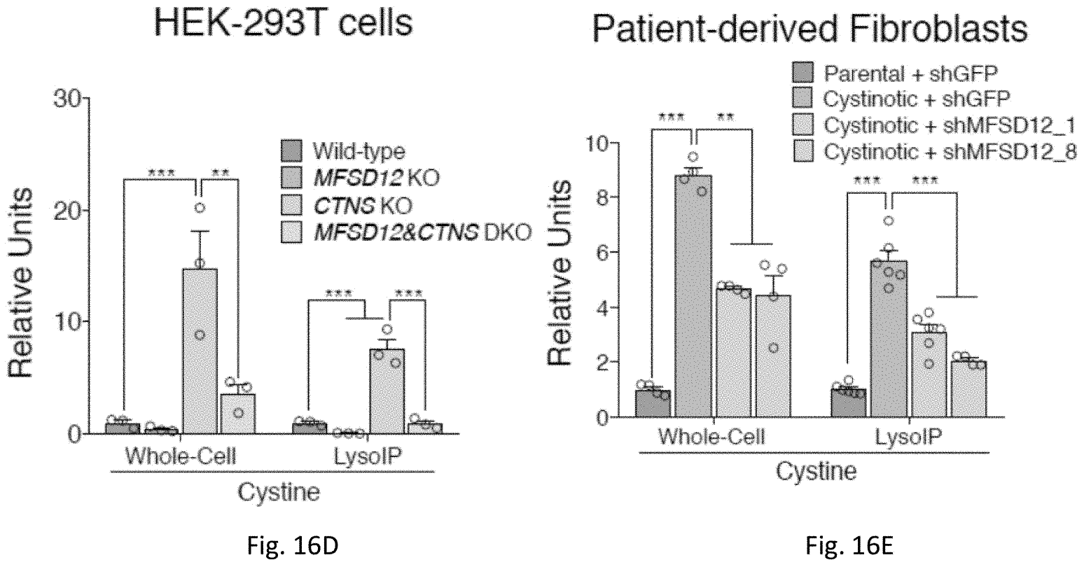

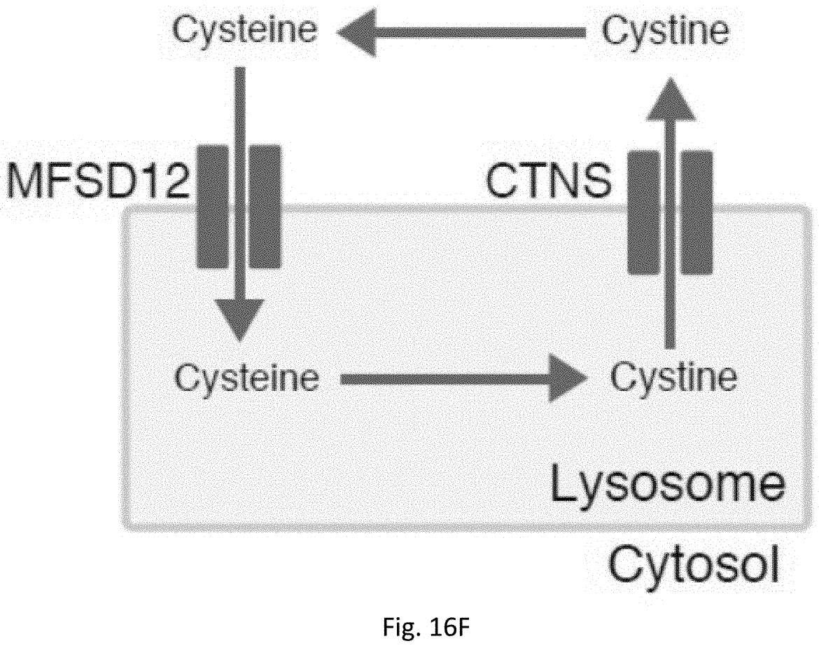

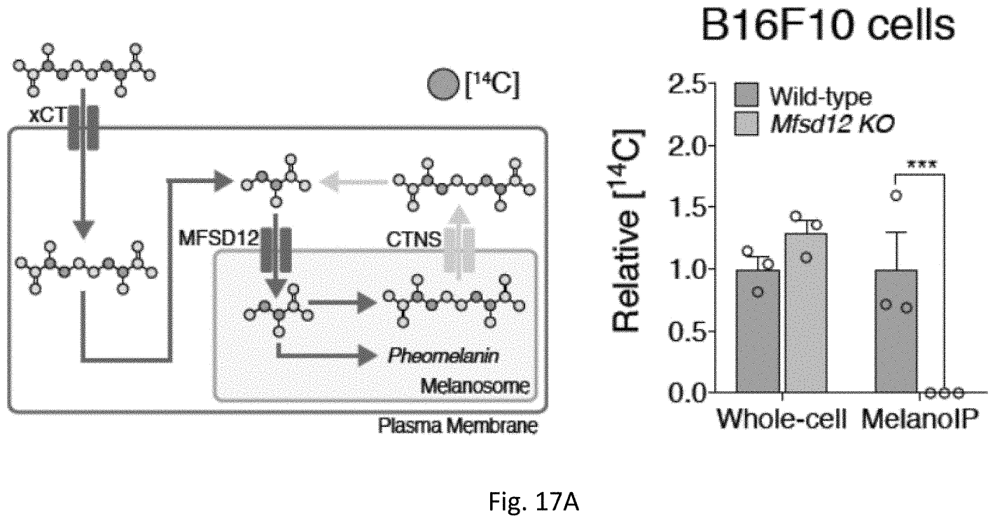

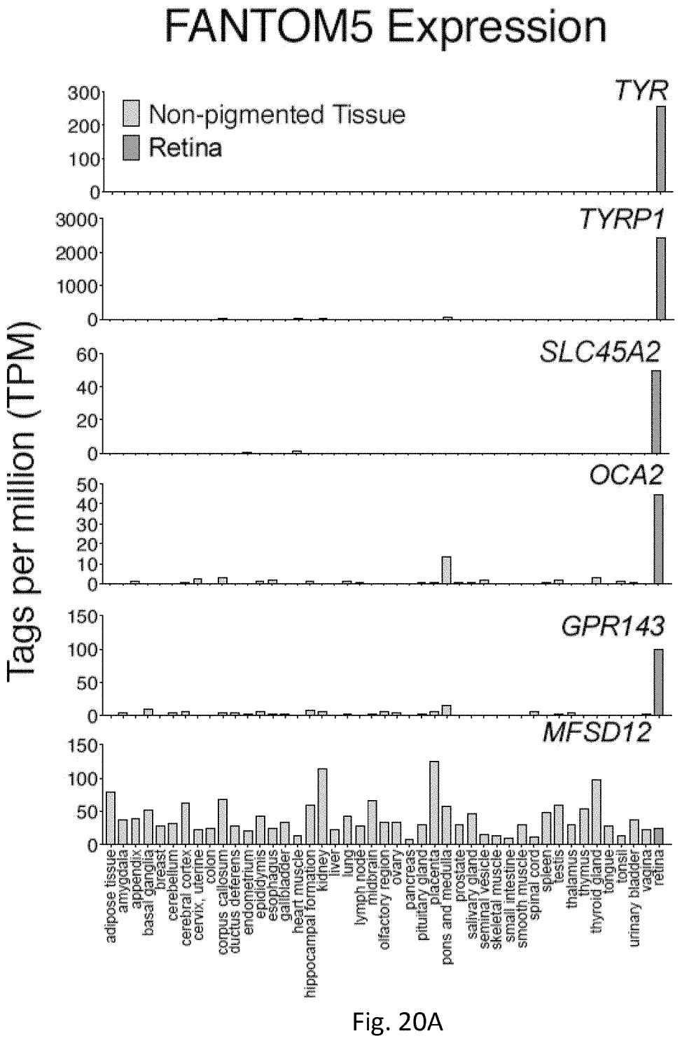

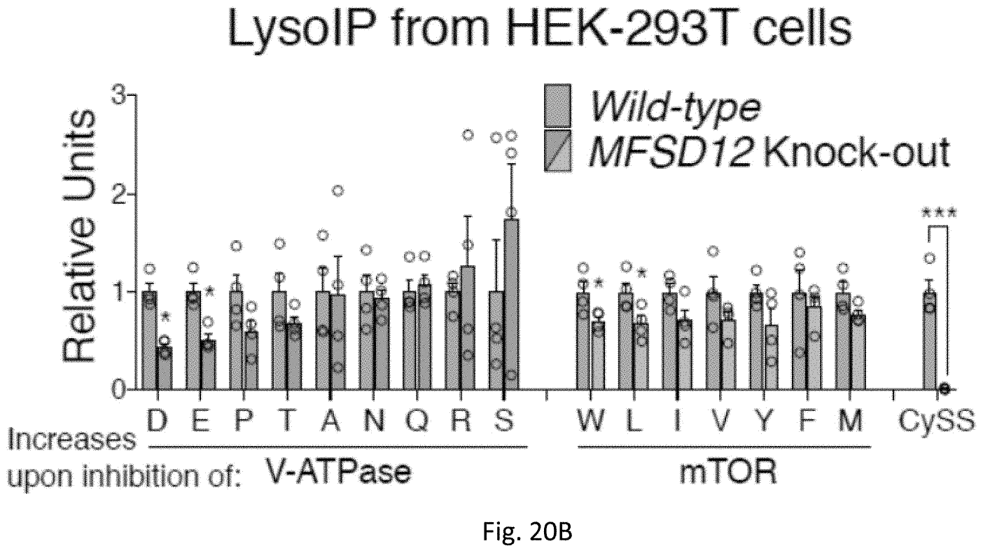

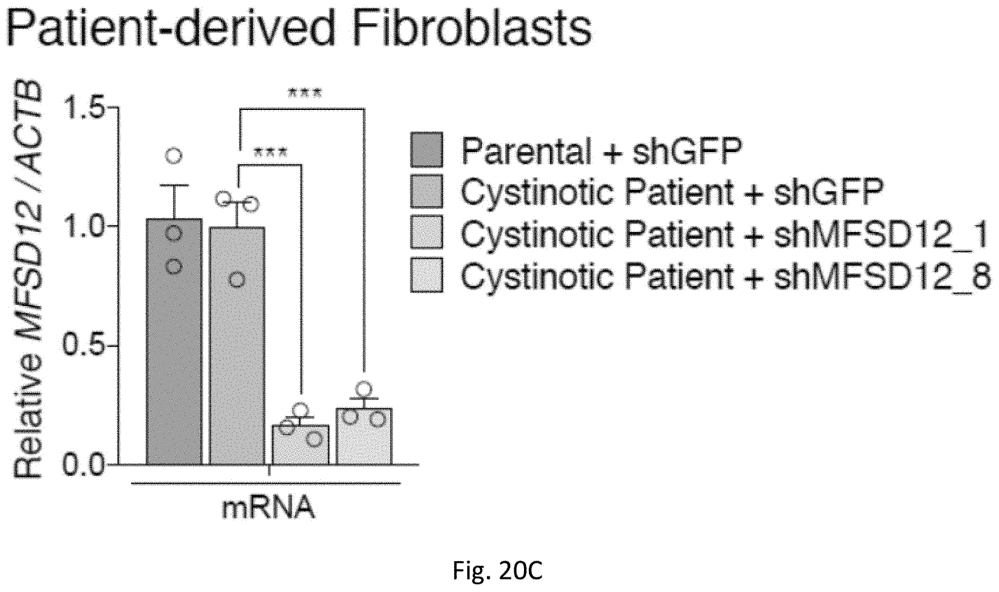

Many genes involved in pigmentation, such as TYR and SLC45A2, are only expressed in pigmented tissues and cells, but MFSD12 is expressed body-wide (FIG. 16A, FIG. 20) (Kawaji et al., 2017; Uhlén et al., 2015). Consistent with MFSD12 also functioning in non-pigmented cells, lysosomes from HEK-293T cells lacking MFSD12 had strongly reduced levels of cystine as detected by untargeted metabolite profiling (FIG. 16B). Follow-up work showed that MFSD12 loss decreased lysosomal cystine levels over 20-fold, which was rescued by MFSD12 re-expression (FIG. 16C). Importantly, MFSD12 loss did not cause any increases in alanine, proline, or other amino acids that tend to accumulate in lysosomes upon inhibition of the V-ATPase, which is required to acidify the lysosomal lumen (FIG. 20B) (Abu-Remaileh et al., 2017), suggesting that MFSD12 impacts cystine levels independently of an effect on the lysosomal pH. In whole-cell samples from HEK-293T cells, a 2.4-fold decrease in cystine was detected upon MFSD12 deletion, consistent with previous data suggesting that a large fraction of total cellular cystine is in lysosomes (Pisoni, 1990). In contrast to melanosomes in which cysteine was undetectable, cysteine could be readily measured in lysosomes from HEK-293T cells and found that MFSD12 loss reduced it by 41-fold (FIG. 16C). As with melanosomes, cysteine transport into isolated lysosomes was previously reported (Pisoni, 1990). The data disclosed herein point to MFSD12 as a long-sought component of such an import system.

While how cysteine enters melanosomes and lysosomes is unknown, it is well appreciated that cystine effluxes out of these organelles through the transporter CTNS (cystinosin) (Gahl et al., 1982; Jonas et al., 1982; Town et al., 1998). Humans with biallelic loss-of-function mutations in CTNS manifest cystinosis, a debilitating lysosomal storage disorder driven by cystine accumulation in tissues across the body, particularly the kidney (Gahl et al., 2002; Town et al., 1998). It was reasoned that a major source of lysosomal cystine is the oxidation of cysteine brought into lysosomes in an MFSD12-dependent fashion. To test this possibility, cystine in whole-cell and lysosomal samples from HEK-293T cells lacking MFSD12, CTNS, or both were measured. Loss of CTNS increased cystine levels 15- and 8-fold in whole-cell and lysosomal samples, respectively (FIG. 16D). The concomitant loss of MFSD12 reversed these increases, normalizing them to approximately the levels seen in wild-type cells (FIG. 16D). The higher amount of cystine in lysosomes from the double knock-out cells compared to those just lacking MFSD12 suggests that other processes, such as lysosomal proteolysis or vesicular traffic, also contribute to maintaining lysosomal cystine levels, albeit to much smaller extents than MFSD12. Consistent results were obtained using RNAi to reduce MFSD12 levels in fibroblasts derived from patients with cystinosis (FIG. 16E and FIG. 20B). The reduction in lysosomal cystine was more modest than what was observed in the knock-out HEK-293T cells, likely because the RNAi reduced the MFSD12 mRNA by only 75-85% so that residual MFSD12 protein remains. These data are consistent with MFSD12 functioning upstream of CTNS (FIG. 16F).

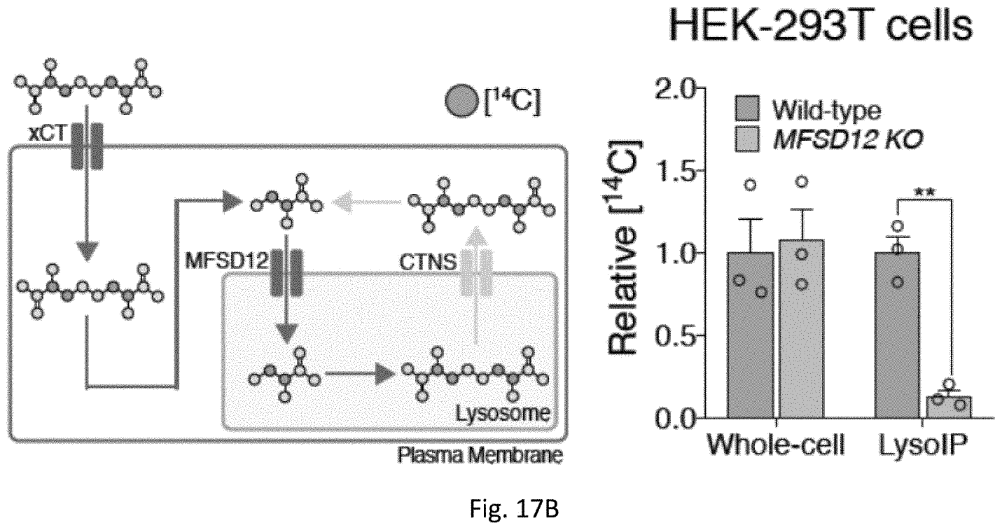

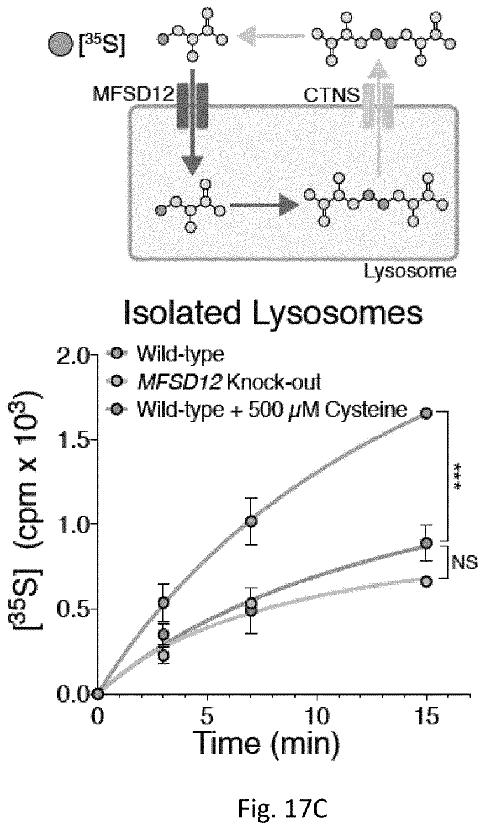

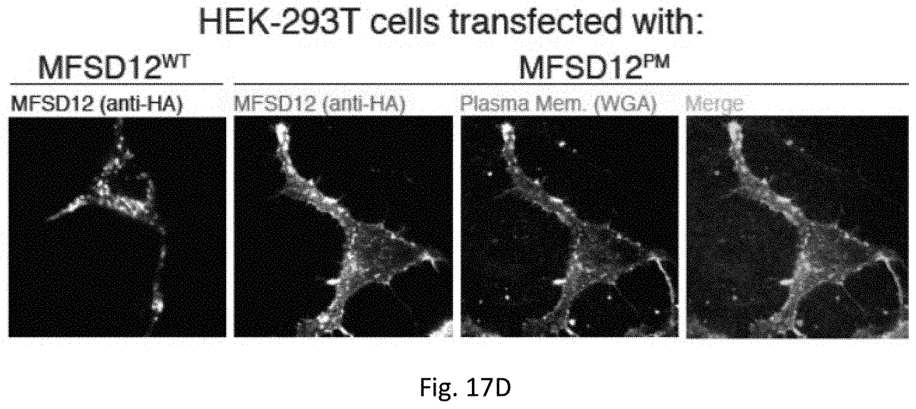

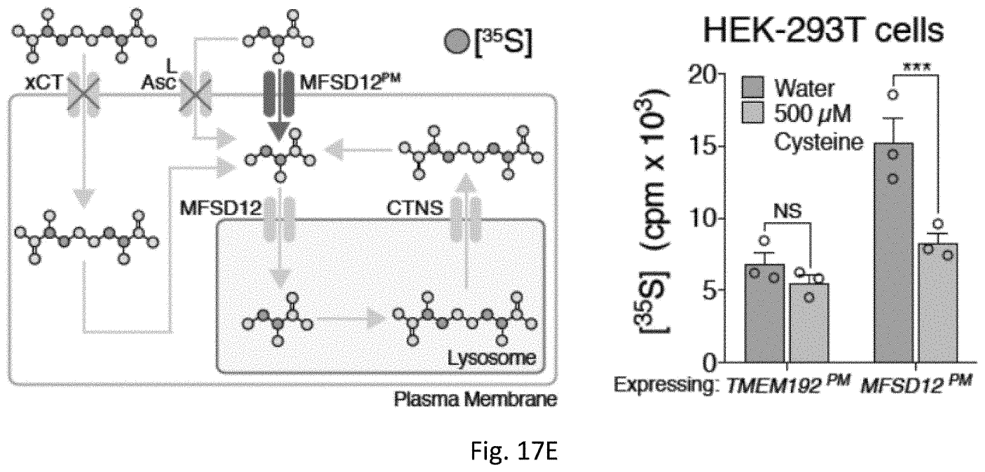

Mammalian cells acquire most of their cysteine by importing extracellular cystine via the xCT plasma membrane transporter and rapidly reducing it to cysteine in the cytosol (Sato et al., 1999). Loss of MFSD12 had no impact on the plasma membrane transport of [¹⁴C]-cystine in B16F10 or HEK-293T cells (FIGS. 17A-17B). In contrast, in cells lacking MFSD12 and given [¹⁴C]-cystine in extracellular buffer, the amount of [¹⁴C] recovered in melanosomes and lysosomes captured via rapid immunoprecipitation methods was severely blunted. It was undetectable over background in the case of melanosomes and more than 7.5-fold lower in lysosomes (FIGS. 17A-17B). While these results were consistent with MFSD12 being necessary for the uptake of cysteine into melanosomes and lysosomes, it could not be ruled out that the cysteine derived from the labeled cystine was converted into something else before its MFSD12-dependent import into these organelles.

Thus, to directly test the necessity of MFSD12 in lysosomal cysteine import differential centrifugation was used to isolate large quantities of lysosomes from wild-type and MFSD12 knock-out HEK-293T cells. Lysosomes containing MFSD12 sequestered about 3-fold more [³⁵S]-cysteine than those without it during a 15-minute transport assay (FIG. 17C). Importantly, the addition of 500 μM unlabeled cysteine was sufficient to reduce the [³⁵S] signal in lysosomes having MFSD12 to the same level as those in lysosomes lacking it (FIG. 17C). This suggests that the transport assay has a high background caused by low affinity interactions of the highly reactive [³⁵S]-cysteine and that there is no other high-affinity cysteine import system in lysosomes. Collectively, the results indicate that MFSD12 is necessary for cysteine import into melanosomes and lysosomes.

As a test of sufficiency, it was asked if MFSD12 could promote the transport of cysteine between a different pair of compartments, namely from the extracellular space into the cytosol. A single dileucine motif (LL253-254) was identified in MFSD12 that when mutated reduced its localization to lysosomes and re-directed it to the plasma membrane (FIG. 17D). In a buffer containing inhibitors of other systems with the potential to transport cysteine and cystine, HEK-293T cells expressing plasma membrane-localized MFSD12 had an enhanced rate of [³⁵S]-cysteine uptake compared to cells expressing a plasma membrane-localized mutant of the lysosomal transmembrane protein TMEM192 (FIG. 17E) (Behnke et al., 2011). Critically, unlabeled cysteine completely competed MFSD12-induced cysteine transport (FIG. 17E). These results suggest that MFSD12 is sufficient to transport cysteine.

This work indicates that the pigmentation protein MFSD12 is necessary and likely sufficient for the transport of cysteine into melanosomes and lysosomes, processes that were previously described (Pisoni, 1990; Potterf et al., 1999), but for which no protein component of the import system had been convincingly identified. In melanosomes, MFSD12 provides cysteine for the production of the cysteinyldopas used in pheomelanin synthesis, giving a molecular explanation for how genetic variation in MFSD12 influences human pigmentation (Adhikari et al., 2019; Crawford et al., 2017). In lysosomes, cysteine is thought to promote the activity of lysosomal proteases (Lloyd, 1986; Mego, 1984), and these findings provide a potential explanation for why MFSD12 has scored in a genetic screen that indirectly assays lysosomal function (Tsui et al., 2019). Cysteine might represent a primary reducing ‘currency’ in the lysosome (Lloyd, 1986), and the identification of MFSD12 provides a handle to study the cysteine to cystine cycle in lysosomes in vivo. MFSD12 inhibitors may represent a new therapeutic class for the treatment of cystinosis, a disease for which cysteamine has remained the standard of care since it was developed in 1976 (Gahl et al., 1987; Thoene et al., 1976). Given the function of MFSD12 in melanosomes, such inhibitors may also darken skin and thus function as sunless tanning agents (Mujahid et al., 2017). It is striking that while the core functions of melanosomes and lysosomes are so different, MFSD12 plays important roles in both. This work provides a roadmap for how the MelanolP method along with untargeted metabolite profiling can be used to deorphan the function of other melanosomal proteins with roles in human pigmentation.

### METHODS

### Cell Culture

HEK-293T cells, B16F10 cells, and patient-derived fibroblasts were propagated in IMDM (Gibco) supplemented with 10% heat-inactivated fetal calf serum (FCS, Gibco). SKMEL30 cells were propagated in RPMI (Gibco) supplemented with 10% FCS (Gibco). Suspension cultures of HEK-293T and B16F10 cells were generated by transferring adherent cells to FreeStyle media (Gibco) supplemented with 1% FCS in a rotating shaker. All medias were supplemented with penicillin/streptomycin (50 U/mL, Gibco).

Virus Production and Generation of Cells Stably-Expressing cDNAs

Lentiviruses were produced by co-transfection of HEK-293T cells with a target lentiviral transfer vector (1 μg), pCMV-VSV-G (100 ng, Addgene #8454), and pCMV-dR8.2 (900 ng, Addgene #8455) plasmids. Retrovirus was generated by substituting pCMV-dR8.2 with pCL-Eco (Addgene #12371). Virus containing supernatants were harvested forty-eight hours post-transfection and stored at −80° C. For infection, ˜1 million cells were incubated with 50-1000 μL viral supernatant, 10 μg/ml polybrene, and 2 mL of culture media in a 6-well plate before spinning at 1,200×g for 45 minutes at 37° C. Infected cells were passaged into media containing selection agent 36-48 hours post-transduction. Selection was maintained for 3 to 5 days before use.

### Knock-Out Cell Line Generation

Knock-out cell lines were generated by infection with lentiCRISPRv2-Opti (Addgene # Pending) vectors encoding single guide RNAs (sgRNAs) directed against early exonic regions of genes of interest. Clonal knock-out cell lines were isolated through fluorescence-activated cell sorting and frameshifted alleles were confirmed by deep-sequencing of each locus. Double-knockouts were isolated by the same process, starting with clonal single knock-out cells. The following oligonucleotides were used for sgRNA cloning and include cloning overhangs for ligation after BsmBI digest of lentiCRISPRv2-Opti vector:

Tyr (Mouse):
Sense:
(SEQ ID NO: 1)
caccgATGGGTGATGGGAGTCCCTG
Anti-sense:
(SEQ ID NO: 2)
aaacCAGGGACTCCCATCACCCATc
Mfsd12 (Mouse):
Sense:
(SEQ ID NO: 3)
caccGAAGCTCAGCCGCGCGGCGA
Anti-sense:
(SEQ ID NO: 4)
aaacTCGCCGCGCGGCTGAGCTTC
MFSD12 (Human):
Sense:
(SEQ ID NO: 5)
caccgCAAAGGCCAGGATCACCAGG
Anti-sense:
(SEQ ID NO: 6)
aaacCCTGGTGATCCTGGCCTTTGc
CTNS (Human):
Sense:
(SEQ ID NO: 7)
caccGATTTCAAAAGTGATCACCA
Anti-sense:
(SEQ ID NO: 8)
aaacTGGTGATCACTTTTGAAATC

Generation of cDNA Constructs

HA-MelanoTag (Addgene # Pending) and myc-MelanoTag (Addgene # Pending) lentiviral constructs: the GPR143 cDNA was isolated via PCR from a cDNA library generated from SKMEL30 cells and cloned directly into pLJC6 (Addgene #104435) containing a contiguous reading frame with c-terminal mScarlet-3xHA or mScarlet-myc. pRK5-HA-MFSD12 (Addgene # Pending): A codon-optimized MFSD12 nucleotide sequence was synthesized as a gBlock from IDT and cloned into pRK5 (Addgene #46326) with a c-terminal TEV-3xFLAG-3xHA tag. MFSD12 mutants (LL253-254AA) were generated with this construct using an adapted QuikChange protocol. pRK5-HA-TMEM^{PM}: TMEM192 from a LysoTag lentiviral vector (Addgene #102930) was transferred to pRK5 (Addgene #46326) and mutagenized with an adapted QuikChange protocol. pCHA1.1-MFSD12-V5 (Addgene # Pending): MFSD12 coding sequence was transferred from pRK5 to the pLJC6 (Addgene #104435) variant pCHA1 with c-terminal V5-tag encoded on its 3′ primer.

The recoded MFSD12 nucleotide sequence is:

(SEQ ID NO: 9)
ATGGGTCCTGGTCCACCTGCCGCTGGCGCGGCTCCA
TCTCCACGGCCCCTGAGTCTCGTAGCCCGGTTGTC
CTATGCCGTGGGCCATTTCCTTAATGACCTTTGTG
CCAGTATGTGGTTTACCTACTTGTTGCTTTATCTC
CATAGCGTGAGAGCTTATTCTTCTCGAGGAGCGGG
TTTGCTCCTGTTGCTGGGCCAAGTTGCAGATGGCC
TGTGCACCCCTCTTGTGGGTTATGAGGCCGATAGA
GCGGCAAGTTGCTGTGCAAGGTATGGTCCGCGAAA
GGCATGGCATTTGGTTGGCACCGTATGCGTACTTC
TTTCTTTCCCATTTATCTTCTCTCCATGTCTCGGC
TGCGGCGCAGCCACCCCCGAATGGGCTGCTTTGTT
GTATTACGGACCTTTCATCGTAATATTCCAATTCG
GCTGGGCGTCAACGCAAATTAGCCATCTCAGTCTC
ATTCCGGAACTTGTTACTAATGATCACGAAAAAGT
AGAGCTTCGCTATGCCTTCACCGTTGTAGCCAACA
TAACGGTAcACGGCGCTGCCTGGCTTCTCTTGCAC
CTCCAGGGGAGCTCAAGGGTCGAGCCAACGCAGGA
TATTTCAATATCAGACCAGCTCGGTGGTCAGGATG
TTCCGGTGTTCCGGAATCTGAGTTTGCTCGTCGTC
GGAGTTGGAGCTGTCTTCAGCCTTCTTTTCCACCT
TGGAACAAGGGAACGCAGACGGCCTCACGCTGAAG
AGCCAGGTGAGCACACTCCGCTTCTGGCCCCTGCT
ACAGCGCAACCCTTGCTTCTCTGGAAGCATTGGCT
GAGAGAACCCGCTTTCTATCAAGTGGGCATACTGT
ATATGACAACGAGGCTTATTGTGAATCTGAGTCAA
ACCTATATGGCCATGTATTTGACATATTCTCTTCA
CTTGCCTAAAAAGTTCATCGCCAcAATTCCGCTTG
TTATGTATCTGAGTGGTTTCTTGAGTAGCTTTCTG
ATGAAGCCGATCAACAAGTGCATTGGACGGAACAT
GACGTACTTCAGTGGCCTTTTGGTCATCCTGGCTT
TTGCAGCATGGGTTGCTCTTGCGGAGGGCCTGGGA
GTAGCAGTGTATGCGGCTGCTGTTCTGTTGGGGGC
CGGGTGTGCAACAATCCTCGTGACGTCCCTTGCGA
TGACGGCAGATCTGATTGGGCCTCACACGAACTCC
GGAGCCTTCGTTTACGGTTCTATGTCCTTCTTGGA
CAAGGTTGCTAATGGGCTTGCCGTGATGGCAATTC
AATCCCTTCACCCGTGCCCTTCTGAGTTGTGCTGC
AGAGCGTGTGTGTCCTTTTATCATTGGGCTATGGT
CGCTGTGACGGGTGGAGTAGGGGTGGCAGCAGCCC
TCTGCCTCTGTAGTCTTTTGCTGTGGCCGACTAGG
CTTCGCCGCTGGGACCGGGACGCCCGCCCG

### RNAi-Mediated Knock-Down Cell Lines

Constructs for shRNA-mediated knock-down were accessed via the Broad Institute RNAi Consortium and were pLKO.1 lentiviral backbones with the following targeting sequences:

shMFSD12_.1:
(SEQ ID NO: 10)
CCCATTCTCAACTCTAATCCA
shMFSD12_.8:
(SEQ ID NO: 11)
CTTCTTGTCCTCCTTCCTCAT

Knock-down was quantified by qPCR. RNA was isolated via RNAeasy Plus Micro Kit (Qiagen) and cDNA was generated with qScript cDNA Supermix (Quantabio). 100 ng RNA equivalents of cDNA were assayed for MFSD12 and ATCB transcript levels with SYBR green qPCR mix (Roche), utilizing the following primer sets:

qPCR_MFSD12_F:
(SEQ ID NO: 12)
TCCACACAGATCTCCCACCT
qPCR_MFSD12_R:
(SEQ ID NO: 13)
GCCGTAGACGGTGATGTTG
qPCR_ACTB_F:
(SEQ ID NO: 14)
GGTTCCGCTGCCCTGAGG
qPCR_ACTB_R:
(SEQ ID NO: 15)
GAAGGTAGTTTCGTGGATGCC

### Immunofluorescence

Immunofluorescence-compatible antibodies to LAMP2 (sc-18822) and TYRP1 (sc-166857) were from Santa Cruz Biotechnology; HA (CST-3724) was from Cell Signaling Technology. Fluorophore-labeled secondary antibodies were from Thermo Fisher Scientific. For B16F10 cells, 10,000 cells were plated in a 24-well plate onto glass coverings and treated with 10 μM forskolin (LC Labs) the next day. The Immunofluorescence assay was performed 48 hours after plating. For HEK-293T cells, 10,000 cells were plated onto fibronectin-coated glass coverings in a 24-well plate and transfected with 50 ng plasmid and 1 μg salmon sperm carrier DNA with polyethylenimine (PEI, 3 μg per 1 μg plasmid). The immunofluorescence assay was performed 48 hours after transfection. Cells were washed with ice cold PBS and fixed in 4% paraformaldehyde in Phosphate Buffered Saline (PBS, Gibco) for 15 minutes at room temperature before rinsing three times with PBS. Fixed cells were then incubated in Blocking Buffer (5% BSA with 0.1% Tween-20 in PBS) for 1 hour at room temperature. Primary antibodies were diluted 1:400 in Blocking Buffer and applied to cells for 1 hour at room temperature. Cells were then washed three times with PBS before incubation with secondary antibodies, diluted 1:400 in Blocking Buffer, for 1 hour at room temperature. Cells were washed three times with PBS and mounted in Vectashield mounting media. For wheat germ agglutinin (WGA) labeling, 5 μg/mL Alex Fluor 594 WGA conjugate (Thermo Fisher) was incubated on live cells 10 minutes before fixation. Cell images were captured with a 63×objective lens mounted on a spinning-disk confocal microscope (Perkin Elmer). Images were processed and prepared in Fiji.

### Rapid Organellar Immunoprecipitation

For B16F10 cells, 3 million MelanoTag-expressing cells were plated per 15 cm plate and treated with 10 μM forskolin (LC Labs) the next day. Two days later, cells were harvested for MelanolP. For SKMEL30 cells, 10 million cells were plated per 15 cm plate and harvested two days after plating. The cell culture media was exchanged two hours before cell harvest. Cells were washed and harvested by scraping in ice cold KPBS (136 mM KCl, 10 mM KH₂PO₄, pH 7.25 adjusted with KOH)(Chen et al., 2016) before pelleting by centrifugation at 2,000×g for 1 minutes at 4° C. Pellets were resuspended in 200 μL ice cold KPBS and 10 μL of the cell suspension was taken as a whole-cell control. Cells were homogenized with a hand-held, rotary pestle (Kimble-Chase) for 45 seconds before the further addition of 800 μL of KPBS and centrifugation at 3,000×g for 2 minutes at 4° C. Supernatants were transferred into new 1.5 mL tubes containing 100 μL of KBPS-washed anti-HA magnetic beads (Thermo Fisher) and incubated rocking for 4 minutes at 4° C. Beads were washed 3 times with 1000 μL ice cold KPBS, transferring to a new Eppendorf tube each time. Whole-cell and immunoprecipitated samples were then lysed in 50 μL Lysis Buffer (40 mM HEPES, pH 7.4; 1% Triton-X100; 2.5 mM MgCl₂; 10 mM (3-glycerol phosphate, 10 mM pyrophosphate, and Complete EDTA-free Protease Inhibitor Cocktail (Roche)) for protein analysis, or 50 μL Extraction Buffer (80% methanol v/v supplemented with 500 nM internal extraction standards (Cambridge Isotope Laboratories, MSK-A2-1.2)) for metabolite analyses. For LysolP experiments, the same procedure was followed as above, but with 10 million LysoTag-expressing (Addgene #102930 and #104435) HEK-293T cells plated into a 15 cm dish one day before immunoprecipitation and without forskolin treatment.

### Immunoblotting

Immunoblotting antibodies to LAMP2 (sc-18822), TYRP1 (sc-166857), Rab38 (sc-390176), and PMEL (sc-377325) were from Santa Cruz Biotechnology; Vdac1 (CST-4661), Calreticulin (CST-12238), Golgin-97 (CST-13192), Erk1/2 (CST-137F5) were from Cell Signaling Technologies; Lamp1 (1D4B) was from the Developmental Studies Hybridoma Bank at the University of Iowa. Cells and organelles, immobilized on beads, were lysed on ice in Lysis Buffer (40 mM HEPES pH 7.4, 1% Triton X-100, 2.5 mM MgCl₂ and Complete EDTA-free Protease Inhibitor Cocktail (Roche)). Cell lysates were clarified by spinning 17,000×g for 8 minutes at 4° C. while organellar lysates were cleared of magnetic beads before adding Laemelli buffer. Lysates were resolved by SDS-PAGE at 120 V. Proteins were transferred for 2 hours at 40 V to PVDF membranes. Membranes were blocked with 5% nonfat dry milk in TBST (Tris-buffered saline with 0.1% Tween-20) before incubation with primary antibodies, diluted 1:1000 in 5% BSA in TBST. Membranes were washed three times in TBST before incubation in 1:3000 species specific HRP-conjugated antibody (Cell Signaling Technologies). Membranes were washed again three times in TBST before visualization with ECL (Thermo Fisher) substrate.

### Tissue Expression

FANTOM5 expression tags were accessed via the Human Protein Atlas at the following address: proteinatlas.org/about/assays+annotation#fantom (Kawaji et al., 2017; Uhlén et al., 2015).

### Polar Metabolite Profiling by LC/MS

LC/MS analysis of polar metabolite content of whole-cell and organellar isolations has been described before (Chen et al., 2016). LC/MS-based analyses were conducted on a QExactive benchtop orbitrap mass spectrometer equipped with an Ion Max source and HESI II probe, which was coupled to a Dionex UltiMate 3000 ultra-high performance liquid chromatography system (Thermo). External mass calibration was performed using a standard calibration mixture every 7 days.

Microliter volumes (generally 2.5 μL) of sample were injected into a SeQuant ZIC-pHILIC Polymeric column (2.1×150 mm; MilliporeSigma) connected with a guard column (2.1×20 mm; MilliporeSigma). Both analytical and guard columns were of 5 μm particle size. Column oven and autosampler tray were held at 25° C. and 4° C., respectively. Mobile Phase A consisted of 20 mM ammonium carbonate, 0.1% ammonium hydroxide. Mobile Phase B was acetonitrile. The flow rate was 0.150 ml minutes-1 with the following gradient programming: (1) 0-20 minutes: linear gradient from 80% to 20% B; (2) 20-20.5 minutes: linear gradient from 20% to 80% B; (3) 20.5-28 minutes: hold at 80% B.

The mass spectrometer was operated in full scan, polarity switching mode with the spray voltage set to 3.0 kV, the heated capillary held at 275° C., and the HESI probe was held at 350° C. Sheath gas flow was set to 40 units, and the auxiliary gas flow was set to 15 units. The MS data acquisition was performed in a range of 70-1000 m/z, with the resolution set to 70,000, the AGC target at 106, and the maximum injection time at 20 msec.

### Metabolomics Data Processing and Analyses

Untargeted metabolic analysis was performed with CompoundDiscoverer v2.0. MS1 data were collected on individual samples, while representative samples, pooled according to genotype, were used to collect ddMS2 data to aid metabolite identification.

Follow-up metabolite identification and quantification were performed by extracting ion chromatograms using XCalibur v4.0 (Thermo Fisher) with a mass tolerance of 5 p.p.m and referencing an in-house library of authentic standards. For relative quantification, the raw peak area for each metabolite was divided by the raw peak area of the relevant isotope-labeled internal standard to calculate the relative abundance.

Melanin Intermediate Validation and Detection ‘Melanin synthesis intermediate’ in untargeted analysis (FIG. 14D) was annotated by CompoundDiscoverer v2.0 using ddMS2 fragmentation data. A dihydroxyindolequinone (DHI) chemical standard (Cayman) was used for follow-up compound validation. When dissolved in water, this standard contained two species with m/z values consistent with DHI and indole-5,6-quinone (Prota, 1992). Consistent with this, indole-5,6-quinone is known to be generated from the spontaneous oxidation of DHI. As this annotated indole-5,6-quinone species was more readily detectable in biological samples, it was quantified here as the ‘Melanin Intermediate’ (FIG. 14E).
Cysteine Measurements with Ellman Labeling

Whole-cell and immunopurified samples were extracted in 25 μL 80% v/v methanol without internal standards. An internal control of 100 nM [U-¹³C, ¹⁵N]-cysteine (Cambridge Isotopes) was added to each sample before the addition of a 1:1 volume of 10 mM solution of Ellman's reagent (Thermo Fisher) prepared in 80% v/v methanol. Samples were incubated on ice for 1 hour before storage at −80° C. Samples were run as above with a targeted selected ion monitoring scan (tSIM) in positive mode centered on m/z 319.00530 and m/z 323.01240 to increase signal for cysteine-TNB and [U-¹³C, ¹⁵N]cysteine-TNB, respectively.

Cysteinyldopas Measurements with Solid Phase Extraction

The cysteinyldopa extraction protocol was adapted from previously described methods for measuring cysteinyldopas in serum (Martin et al., 2007). Cells were plated under the same conditions used for MelanolP protocols. After scraping and washing, cells were lysed via rotary homogenizer (Kimble-Chase) in 0.1 N HCl in water before spinning 18,000×g for 5 minutes at 4° C. 100 nM [¹⁵N]-phenylalanine (Cambridge Isotopes) was added to lysate supernatant as an internal control. MCX cartridges (1 mL, Oasis) were conditioned with 1.0 mL of 100% methanol then 1.0 mL of 0.1 N HCl before applying lysate supernatant and washing with 1.0 mL of 0.1 N HCl then 1.0 mL of 100% methanol. Elution was performed with an 80:10:10 v/v/v mixture of methanol/water/˜30% ammonia with 0.1% ascorbic acid (MilliporeSigma) added as an antioxidant. Samples were acidified after elution with 100 μL formic acid and dried under nitrogen stream before storage at 80° C. Before analysis, extracts were resuspended in 250 μL 0.1% formic acid with 0.1% ascorbic acid and debris were cleared by centrifugation with 18,000×g for 5 minutes at 4° C. before LC/MS analysis.

Detection of cysteinyldopas by LC/MS was performed on the same instrumentation as above, with the following modifications: A 10 μL sample volume was injected onto an Atlantis Express dC18 HPLC column (2.7 μM×150 mm×2.1 mm; MilliporeSigma). Column oven and autosampler tray were held at 20° C. and 4° C., respectively. Mobile Phase A consisted of 0.1% formic acid in water. Mobile Phase B was 0.1% formic acid in 100% methanol. The flow rate was 0.250 ml minutes⁻¹ with the following gradient programming: 0-12 minutes: linear gradient from 0% to 10% B. The mass spectrometer was operated in positive with an MS data acquisition range from 100-350 m/z.

Cysteinyldopa detection was validated with standards synthesized from an adapted protocol for cysteinyldopa synthesis (Ito and Prota, 1977). L-dopa (8.5 mM) and cysteine (17.0 mM) were prepared fresh in 60 mL of 50 mM sodium phosphate buffer, pH 6.8. Reactions were run with the addition of ˜250 U of mushroom tyrosinase (MilliporeSigma), stirring in well aerated conditions at room temperature. Aliquots were quenched by dilution 1:10 in 1 N HCl. After quenching, standards were extracted and analyzed like biological samples.

### Hexosaminidase Assay

Samples were diluted multiple times to ensure signal was quantified within a linear range. Each analyte was then diluted 1:10 in Substrate Solution (90 mM potassium acetate, pH 5.0; 2 mM p-nitrophenyl hexosaminidase substrate; 0.5% Triton X-100) and briefly vortexed to induce lysis before incubation for 30 minutes at 37° C. Samples were quenched with 1 part 0.25 M NaOH to 2 parts of the substrate solution. Absorbance was quantified at 405 nm.

Cellular Cystine Uptake Assays 50 million suspension cells were washed twice with HBSS, and resuspended in 200 μL HBSS. Upon addition of 1.2 μM cystine (Perkin-Elmer), cell suspensions were mixed and incubated at 37° C. with periodic mixing. Cells were then washed twice in ice cold KPBS and organelles were isolated via organellar immunoprecipitation protocols as detailed above. For background quantification samples, 1% Triton X-100 was added to KBPS during the immunoprecipitation and washing steps. Beads were resuspended in 1% Triton X-100 and water and pipetted directly into scintillation fluid before reading scintillation signal (TriCarb T9000TR, Perkin-Elmer). For lysosomal cellular cystine uptake assays, aliquots of whole-cell and immunoprecipitated samples were taken and measured by hexosaminidase assay. Hexosaminidase assay values were used to normalize scintillation counts of cellular input and lysosomal capture.

### Lysosome Uptake Assays

Lysosomal purification protocols were adapted from previous protocols (Graham, 2001). 500 million suspension-adapted HEK-293T cells were spun down, washed in ice-cold PBS, and resuspended in 15 mL homogenization buffer (250 mM sucrose; 20 mM HEPES, pH 7.4; 1 mM EDTA) supplemented with protease inhibitor tablets (Roche). Cells were dounced 10 times in a lose fitting glass dounce and the resulting lysate was centrifuged at 1,500×g for 10 minutes at 4° C. After transferring the supernatant, 15 mL homogenization buffer was used to resuspend the pellet, and douncing and centrifugation were repeated. Supernatants from three successive rounds of homogenization and centrifugation were pooled and centrifuged a final time before transferring ‘post-nuclear supernatant’ to the subsequent step. ‘Post-nuclear supernatant’ was centrifuged at 20,000×g for 10 minutes at 4° C. to pellet organelles. To further wash organelles, the pellet was resuspended in 45 mL homogenization buffer and spun again at 20,000×g for 10 minutes at 4° C. The resulting pellet or ‘light mitochondrial fraction’ was resuspended in 9% OptiPrep (Thermo Fisher) prepared in Dilution Buffer (250 mM sucrose; 40 mM HEPES, pH 7.4; 2 mM EDTA) and overlaid on top of a discontinuous gradient of 18, 16, 14, 12, and 10% OptiPrep prepared in Dilution Buffer. After centrifugation at 145,000×g for 2 hours at 4° C., 1 mL fractions were taken and assayed for lysosomal activity with a hexosaminidase assay. High activity fractions were diluted 1:1 in homogenization buffer and spun 5,000×g for 10 min at 4° C. to pellet lysosomes before resuspending in Lyso Buffer (KPBS, 125 mM sucrose) with the addition of 3 mM DTT and 1 μM cysteine. Each sample's lysosomal content was normalized with a hexosaminidase assay and samples were equilibrated for 30 minutes on ice. Assays were started with the addition of cold cysteine (35 μM for transport conditions and 500 μM for competition) and 50 μCi/mL (<100 μM) [³⁵S]-cysteine (Perkin-Elmer). Samples were incubated at 30° C. with periodic mixing. Aliquots were taken at indicated time points and passed over glass fiber (GFA) filters (MilliporeSigma), blocked overnight with 1% BSA in PBS. Filters were washed two times in ice-cold PBS, dried, and submerged in scintillation fluid before measuring scintillation counts (TriCarb T9000TR, Perkin-Elmer).

### Plasma Membrane Cellular Uptake Assays

3 million HEK-293T cells were plated in 10 cm dishes. Cells were transfected with 3 μg of plasmid with PEI (3 μg per 1 μg plasmid) and scraped 48 hours post-transfection into ice cold KPBS. After washing twice, cells were resuspended in LysoBuffer supplemented with 2.5 μM Erastin (MilliporeSigma), 4 mM leucine, and 3 mM serine. Assays were started with the addition of cold cysteine (10 μM for transport conditions and 500 μM for competition) and 50 μCi/mL (<100 pM) [³⁵S]-cysteine. Samples were incubated at 30° C. and aliquots were taken at indicated time points. Aliquoted cells were harvested by centrifugation at 1,000×g for 30 seconds at 4° C. and washed twice in ice-cold PBS. Cell pellets were resuspended in water before mixing into scintillation fluid and measuring scintillation counts (TriCarb T9000TR, Perkin-Elmer).

### Data Preparation and Statistics

Displays of quantitative data were prepared in GraphPad Prism 8. Statistical comparisons were via two-tailed unpaired t-tests, performed in Prism. All measurements displayed represent samples generated independently. Immunoblot and immunofluorescence data are representative of experiments repeated at least three times.

### REFERENCES

Abu-Remaileh, M., Wyant, G. A., Kim, C., Laqtom, N. N., Abbasi, M., Chan, S. H., Freinkman, E., and Sabatini, D. M. (2017). Lysosomal metabolomics reveals V-ATPase- and mTOR-dependent regulation of amino acid efflux from lysosomes. Science 358, 807-813.
Adhikari, K., Mendoza-Revilla, J., Sohail, A., Fuentes-Guajardo, M., Lampert, J., Chacón-Duque, J. C., Hurtado, M., Villegas, V., Granja, V., Acunã-Alonzo, V., et al. (2019). A GWAS in Latin Americans highlights the convergent evolution of lighter skin pigmentation in Eurasia. Nat Commun 10.
Basrur, V., Yang, F., Kushimoto, T., Higashimoto, Y., Yasumoto, K., Valencia, J., Muller, J., Vieira, W. D., Watabe, H., Shabanowitz, J., et al. (2003). Proteomic Analysis of Early Melanosomes: Identification of Novel Melanosomal Proteins. J. Proteome Res. 2, 69-79.
Behnke, J., Eskelinen, E.-L., Saftig, P., and Schröder, B. (2011). Two dileucine motifs mediate late endosomal/lysosomal targeting of transmembrane protein 192 (TMEM192) and a C-terminal cysteine residue is responsible for disulfide bond formation in TMEM192 homodimers. Biochemical Journal 434, 219-231.
Bissig, C., Rochin, L., and van Niel, G. (2016). PMEL Amyloid Fibril Formation: The Bright Steps of Pigmentation. Int J Mol Sci 17.
Bloom, J. L., and Falconer, D. S. (1966). ‘Grizzled’, a mutant in linkage group X of the mouse. Genetics Research 7, 159-167.
Bruder, J. M., Pfeiffer, Z. A., Ciriello, J. M., Horrigan, D. M., Wicks, N. L., Flaherty, B., and Oancea, E. (2012). Melanosomal Dynamics Assessed with a Live-Cell Fluorescent Melanosomal Marker. PLOS ONE 7, e43465.
Chen, W. W., Freinkman, E., Wang, T., Birsoy, K., and Sabatini, D. M. (2016). Absolute quantification of matrix metabolites reveals the dynamics of mitochondrial metabolism. Cell 166, 1324-1337.e11.
Crawford, N. G., Kelly, D. E., Hansen, M. E. B., Beltrame, M. H., Fan, S., Bowman, S. L., Jewett, E., Ranciaro, A., Thompson, S., Lo, Y., et al. (2017). Loci associated with skin pigmentation identified in African populations. Science 358.
D'Alba, L., and Shawkey, M. D. (2019). Melanosomes: Biogenesis, Properties, and Evolution of an Ancient Organelle. Physiological Reviews 99, 1-19.
Diment, S., Eidelman, M., Rodriguez, G. M., and Orlow, S. J. (1995). Lysosomal Hydrolases Are Present in Melanosomes and Are Elevated in Melanizing Cells. J. Biol. Chem. 270, 4213-4215.
Gahl, W. A., Bashan, N., Tietze, F., Bernardini, I., and Schulman, J. D. (1982). Cystine transport is defective in isolated leukocyte lysosomes from patients with cystinosis. Science 217, 1263-1265.
Gahl, W. A., Reed, G. F., Thoene, J. G., Schulman, J. D., Rizzo, W. B., Jonas, A. J., Denman, D. W., Schlesselman, J. J., Corden, B. J., and Schneider, J. A. (1987). Cysteamine Therapy for Children with Nephropathic Cystinosis. New England Journal of Medicine 316, 971-977.
Gahl, W. A., Thoene, J. G., and Schneider, J. A. (2002). Cystinosis. N. Engl. J. Med. 347, 111-121.
Graham, J. M. (2001). Isolation of lysosomes from tissues and cells by differential and density gradient centrifugation. Curr Protoc Cell Biol Chapter 3, Unit 3.6.
Ito, S., and Prota, G. M. A. (1977). A facile one-step synthesis of cysteinyldopas using mushroom tyrosinase. Experientia 33, 1118-1119.
Jonas, A. J., Smith, M. L., and Schneider, J. A. (1982). ATP-dependent lysosomal cystine efflux is defective in cystinosis. J. Biol. Chem. 257, 13185-13188.
Kawaji, H., Kasukawa, T., Forrest, A., Carninci, P., and Hayashizaki, Y. (2017). The FANTOM5 collection, a data series underpinning mammalian transcriptome atlases in diverse cell types. Scientific Data 4, 1-3.
Lloyd, J. B. (1986). Disulphide reduction in lysosomes. The role of cysteine. Biochem J 237, 271-272.
Martin, G. B., Chiap, P., Paquet, P., Pierard, G., de Tullio, P., Martin, Y., Rozet, E., Hubert, P., Crommen, J., and Fillet, M. (2007). Development of a mass spectrometry method for the determination of a melanoma biomarker, 5-S-cysteinyldopa, in human plasma using solid phase extraction for sample clean-up. Journal of Chromatography A 1156, 141-148.
Mego, J. L. (1984). Role of thiols, pH and cathepsin D in the lysosomal catabolism of serum albumin. Biochem J 218, 775-783.
Mujahid, N., Liang, Y., Murakami, R., Choi, H. G., Dobry, A. S., Wang, J., Suita, Y., Weng, Q. Y., Allouche, J., Kemeny, L. V., et al. (2017). A UV-Independent Topical Small-Molecule Approach for Melanin Production in Human Skin. Cell Rep 19, 2177-2184.
Pisoni, R. L. (1990). A cysteine-specific lysosomal transport system provides a major route for the delivery of thiol to human fibroblast lysosomes: possible role in supporting lysosomal proteolysis. The Journal of Cell Biology 110, 327-335.
Potterf, S. B., Virador, V., Wakamatsu, K., Furumura, M., Santis, C., Ito, S., and Hearing, V. J. (1999). Cysteine Transport in Melanosomes from Murine Melanocytes. Pigment Cell Research 12, 4-12.
Prota, G. (1992). Melanins and Melanogenesis (New York, N. Y.: Academic). Sato, H., Tamba, M., Ishii, T., and Bannai, S. (1999). Cloning and expression of a plasma membrane cystine/glutamate exchange transporter composed of two distinct proteins. J. Biol. Chem. 274, 11455-11458.
Sturm, R. A. (2009). Molecular genetics of human pigmentation diversity. Human Molecular Genetics 18, R9-R17.
Thoene, J. G., Oshima, R. G., Crawhall, J. C., Olson, D. L., and Schneider, J. A. (1976). Cystinosis. Intracellular cystine depletion by aminothiols in vitro and in vivo. J Clin Invest 58, 180-189.
Town, M., Jean, G., Cherqui, S., Attard, M., Forestier, L., Whitmore, S. A., Callen, D. F., Gribouval, O., Broyer, M., Bates, G. P., et al. (1998). A novel gene encoding an integral membrane protein is mutated in nephropathic cystinosis. Nature Genetics 18, 319-324.
Tsui, C. K., Barfield, R. M., Fischer, C. R., Morgens, D. W., Li, A., Smith, B. A. H., Gray, M. A., Bertozzi, C. R., Rabuka, D., and Bassik, M. C. (2019). CRISPR-Cas9 screens identify regulators of antibody—drug conjugate toxicity. Nat Chem Biol 15, 949-958.
Uhlén, M., Fagerberg, L., Hallström, B. M., Lindskog, C., Oksvold, P., Mardinoglu, A., Sivertsson, Å., Kampf, C., Sjöstedt, E., Asplund, A., et al. (2015). Tissue-based map of the human proteome. Science 347.
Watabe, H., Kushimoto, T., Valencia, J. C., and Hearing, V. J. (2005) April; Chapter 3:Unit 3.14. Isolation of Melanosomes. In Current Protocols in Cell Biology, J. S. Bonifacino, M. Dasso, J. B. Harford, J. Lippincott-Schwartz, and K. M. Yamada, eds. (Hoboken, N. J., USA: John Wiley & Sons, Inc.).
Yan, N. (2015). Structural Biology of the Major Facilitator Superfamily Transporters. Annual Review of Biophysics 44, 257-283.

1. A method of modulating or stabilizing cysteine transport function in a lysosome of a cell, comprising modulating the expression of MFSD12 or the activity of a gene product of MFSD12 in the cell.

2.-6. (canceled)

7. The method of claim 1, wherein the expression of MFSD12 or the activity of a gene product of MFSD12 is decreased, thereby decreasing the level of cysteine in the lysosome in the cell.

8. The method of claim 1, wherein the expression of MFSD12 or the activity of a gene product of MFSD12 is modulated by contacting the cell with an agent.

9.-12. (canceled)

13. The method of claim 1, wherein the expression of MFSD12 or the activity of a gene product of MFSD12 is modulated using gene editing.

14.-15. (canceled)

16. A method of treating or preventing a disease or disorder associated with an aberrant level of cysteine in a lysosome in a cell of a subject, comprising administering to the subject an agent that modulates the expression of MFSD12 or the activity of a gene product of MFSD12.

17. (canceled)

18. The method of claim 16, wherein administration of the agent decreases the level of cysteine in lysosomes of the subject.

19. The method of claim 16, wherein the agent comprises a polypeptide, amino acid, oligonucleotide, lipid, carbohydrate, hybrid molecule, peptide, nucleic acid, or small molecule.

20. The method of claim 19, wherein the agent is a small molecule.

21. The method of claim 19, wherein the agent is a nucleic acid.

22. The method of claim 21, wherein the nucleic acid comprises a siRNA, shRNA, antisense oligonucleotide, aptamer, or random oligonucleotide.

23. The method of claim 16, wherein the expression of MFSD12 or the activity of a gene product of MFSD12 is modulated using gene editing.

24. The method of claim 23, wherein the gene editing comprises CRISPR, TALEN, or ZFN.

25. The method of claim 24, wherein the gene editing comprises CRISPR.

26. The method of claim 16, wherein the disease or disorder is a lysosomal storage disease or disorder.

27. The method of claim 16, wherein the disease or disorder is cystinosis.

28.-77. (canceled)

78. A method of treating or preventing a disease or disorder associated with an aberrant level of cysteine in a melanosome in a cell of a subject, comprising administering to the subject an agent that modulates the expression of MFSD12 or the activity of a gene product of MFSD12.

79. (canceled)

80. The method of claim 78, wherein administration of the agent decreases the level of cysteine in melanosomes of the subject.

81. The method of claim 78, wherein the agent comprises a polypeptide, amino acid, oligonucleotide, lipid, carbohydrate, hybrid molecule, peptide, nucleic acid, or small molecule.

82. (canceled)

83. The method of claim 81, wherein the agent is a nucleic acid.

84. (canceled)

85. The method of claim 78, wherein the expression of MFSD12 or the activity of a gene product of MFSD12 is modulated using gene editing.

86.-98. (canceled)
